(19) United States
(12) Patent Application Publication
(10) Pub. No.: US 2003/0068626 A1
(43) Pub. Date: Apr. 10, 2003

(54) Pin1 as a marker for abnormal cell growth

(75) Inventors: Da Gong Wang, Chestnut Hill, MA (US); Lere Bao, Newton, MA (US)

Correspondence Address: LAHIVE & COCKFIELD, 28 STATE STREET, BOSTON, MA, 02109, US

(73) Assignee: Pintex Pharmaceuticals, Inc., Watertown, MA

(21) Appl. No.: 10/071,747

(22) Filed: Feb. 8, 2002

Related U.S. Application Data
(60) Provisional application No. 60/267,575, filed on Feb. 9, 2001.

(51) Int. Cl. C12Q 1/68; G01N 33/574

(52) U.S. Cl. 435/6; 435/7.23

(57) ABSTRACT

Methods for the use of Pin1 as a marker of abnormal cell growth are disclosed. In one embodiment, the method includes detecting a level of Pin1 to stage an abnormal cell growth, such as breast or prostate cancer. In another embodiment, the method includes evaluating the efficacy of a treatment of an abnormal cell growth, such as cancer, by monitoring the levels of Pin1. In another embodiment, the method includes evaluating the extent of metastasis of abnormal cell growth, such as cancer. The levels of Pin1 can be protein levels or nucleic acid levels.

### RELATED APPLICATIONS

This application claims priority to U.S. provisional Application (Serial No. 60/267,575), filed on Feb. 9, 2001 and entitled “Pin1 as a Marker for Abnormal Cell Growth” the contents of which are incorporated herein in their entirety by reference.

### Government Support

This invention was made, in whole or in part, by grants RO1GM56230 and RO1GM58556 from the National Institutes of Health. The Government has certain rights in the invention.

### BACKGROUND OF THE INVENTION

The increased number of cancer cases reported in the United States, and, indeed, around the world, is a major concern. Currently there are only a handful of detection and treatment methods available for some specific types of cancer, and these provide no absolute guarantee of success. In order to be most effective, these treatments require not only an early detection of the malignancy, but a reliable assessment of the severity of the malignancy.

Cancers can be viewed as a breakdown in the communication between tumor cells and their environment, including their normal neighboring cells. Growth-stimulatory and growth-inhibitory signals are routinely exchanged between cells within a tissue. Normally, cells do not divide in the absence of stimulatory signals or in the presence of inhibitory signals. In a cancerous or neoplastic state, a cell acquires the ability to “override” these signals and to proliferate under conditions in which a normal cell would not.

In general, cancerous cells must acquire a number of distinct aberrant traits in order to proliferate in an abnormal manner. Reflecting this requirement is the fact that the genomes of certain well-studied tumors carry several different independently altered genes, including activated oncogenes and inactivated tumor suppressor genes. In addition to abnormal cell proliferation, cells must acquire several other traits for tumor progression to occur. For example, early on in tumor progression, cells must evade the host immune system. Further, as tumor mass increases, the tumor must acquire vasculature to supply nourishment and remove metabolic waste. Additionally, cells must acquire an ability to invade adjacent tissue. In many cases, cells ultimately acquire the capacity to metastasize to distant sites.

It is apparent that the complex process of tumor development and growth must involve multiple gene products. It is therefore important to define the role of specific genes involved in tumor development and growth, and to identify those genes and gene products that can serve as targets for the diagnosis, prevention and treatment of cancers.

In the realm of cancer therapy it often happens that a therapeutic agent that is initially effective for a given patient becomes, over time, ineffective or less effective for that patient. The very same therapeutic agent may continue to be effective over a long period of time for a different patient. Further, a therapeutic agent that is effective, at least initially, for some patients can be completely ineffective or even harmful for other patients. Accordingly, it would be useful to identify genes and/or gene products that represent prognostic genes with respect to a given therapeutic agent or class of therapeutic agents. It then may be possible to determine which patients will benefit from particular therapeutic regimen and, importantly, determine when, if ever, the therapeutic regime begins to lose its effectiveness for a given patient. The ability to make such predictions would make it possible to discontinue a therapeutic regime that has lost its effectiveness well before its loss of effectiveness becomes apparent by conventional measures.

### SUMMARY OF THE INVENTION

The invention relates to methods of detecting abnormal cell growth in a mammal, comprising assessing the level of Pin1 in a test sample from the mammal, wherein an elevation in the levels of Pin-1 is indicative of abnormal cell growth. In one embodiment, the level of Pin-1 is a protein level. In another embodiment, the level of Pin1 is a nucleic acid level.

Specifically, in one embodiment the invention relates to epithelial test samples such as breast, uterus, ovarian, brain, endometrium, cervical, colon, esophagus, hepatocellular, kidney, mouth, prostate, liver, lung, skin, or testicular epithelial test samples. In another embodiment the test samples can endocrine, e.g., thyroid. In another embodiment, the test sample can be a body fluid sample, such as blood, ascites or brain fluid.

In particular, the invention relates to a method of detecting abnormal cell growth in a mammal, comprising the steps of detecting a level of Pin1 in a test sample and comparing the level of Pin1 in the test sample with a control level, wherein a difference in the level of Pin-1 in the test sample is indicative of abnormal cell growth in the mammal. An elevation in the level of Pin1 compared to the control level is indicative of the presence the abnormal cell growth in the mammal. Methods of the invention can detect abnormal cell growth that is benign or malignant (e.g., oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, liposarcoma abnormal cell growth).

The invention further relates to a method of detecting abnormal cell growth in a mammal by assessing the level of Pin1 protein in a test sample from the mammal, comprising the steps of contacting the test sample with an antibody having specificity for Pin1 under conditions suitable for binding of the antibody to Pin1 thereby resulting in the formation of a complex between the antibody and Pin1; detecting the complex between the antibody and Pin1; and comparing the amount of the complex in the test sample with an amount of a complex in a control sample, wherein an elevation in the amount of the complex between the antibody and Pin1 in the test sample compared to the complex in the control sample is indicative of abnormal cell growth. The antibody can be a polyclonal or a monoclonal antibody and, optionally, detectably labeled. (e.g., radioactive, enzymatic, magnetic, biotinylated and/or fluorescence).

The invention also relates to a method of detecting abnormal cell growth in a mammal, comprising the steps of detecting a level of Pin1 nucleic acid in a test sample; and comparing the level of Pin1 in the test sample with a level of Pin1 in a control sample is indicative of abnormal cell growth.

Another embodiment of the invention relates to a method of determining abnormal cell growth in a mammal, comprising the steps of contacting a test sample obtained from the mammal with a nucleic acid probe to a Pin1 nucleic acid; maintaining the test sample and the nucleic acid probe under conditions suitable for a hybridization; detecting the hybridization between the test sample and the nucleic acid probe; and comparing the hybridization in the test sample from the mammal to a control test sample without abnormal cell growth, wherein an elevation in the hybridization signal in the test sample from the mammal compared to the control sample is indicative of abnormal cell growth. The nucleic acid probe can be optionally labeled with a label comprising a fluorescent, radioactive, and enzymatic label.

In yet another embodiment, the invention relates to a method of determining a stage of abnormal cell growth, comprising assessing a level of Pin1 in a test sample from a mammal. Specifically encompassed by the invention, is a method of staging oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, liposarcoma abnormal cell growth.

The invention also relates to a method of determining a stage of abnormal cell growth in a mammal by assessing the level of Pin1 in a test sample from the mammal, comprising the steps of contacting the test sample with an antibody having specificity for Pin1 under conditions suitable for binding of the antibody to Pin1 thereby resulting in the formation of a complex between the antibody and Pin1; and comparing the amount of the complex in the test sample with an amount of a complex in a control sample, wherein an elevation in the amount of the complex in the test sample compared to the control sample is indicative of the stage of the cancer. In a related embodiment, the invention relates to a monoclonal anitbody specific for Pin1.

Another aspect of the invention is a method of determining a stage of an abnormal cell growth in a mammal, comprising assessing a level of a Pin-1 nucleic acid in a test sample, comprising the steps of performing a polymerase chain reaction with oligonucleotide primers capable of amplifying the Pin1 nucleic acid; detecting a level of amplified nucleic acid fragments of the Pin1 nucleic acid; and comparing the level of amplified nucleic acid fragments in the test sample to a sample comprising varying stages of the abnormal cell growth, wherein the stage of the abnormal cell growth in the mammal is determined.

The invention also relates to a method of determining a stage of abnormal cell growth in a mammal, comprising the steps of contacting a test sample obtained from the mammal with a nucleic acid probe to a Pin1 nucleic acid; maintaining the test sample and the nucleic acid probe under conditions suitable for hybridization; detecting the hybridization between the test sample and the nucleic acid probe; and comparing the hybridization in the test sample from the mammal to a sample comprising varying stages of the cancer, wherein the stage of abnormal cell growth in the mammal is determined.

In still another embodiment, the invention relates to a method of evaluating the efficacy of a treatment (e.g., surgery, radiation, chemotherapy) of abnormal cell growth in a mammal, comprising comparing a level of Pin1 in at least two test samples comprising a first test sample obtained at a first time and a second test sample obtained at a later second time, wherein a decrease in the level of Pin1 between the two test samples indicates the efficacy of the treatment of the abnormal cell growth in the mammal.

The invention also relates to a method of evaluating the extent of metastasis of abnormal cell growth in a mammal comprising assessing the level of Pin1 in a test sample from the mammal.

In another embodiment, the invention relates to a kit for detecting an abnormal cell growth in a mammal comprising one or more reagents for detecting a level of Pin1 in a test sample obtained from the mammal. Specifically encompassed by the invention are kits for detecting breast, uterus, ovarian, brain, endometrium, cervical, colon, esophagus, hepatocellular, kidney, mouth, prostate, liver, lung, skin, endocrine or testicular cancer employing protein or nucleic acid test samples. In particular, kits for Western blotting, imunocytochemistry, radioimmunoassays (RIA) and enzyme linked immunoabsorption assays are kits of the invention. Also included in the invention are kits, wherein the one or more reagents for detecting the abnormal cell growth are used for carrying out a nucleic acid amplification reaction, such as a polymerase chain reaction based assay.

In yet another embodiment, the invention relates to a kit for determining a stage of abnormal cell growth in a mammal comprising one or more reagents for detecting a level of Pin1 in a test sample obtained from the mammal. Specifically encompassed by the invention are kits for staging of abnormal cell growth of breast, uterus, ovarian, brain, endometrium, cervical, colon, esophagus, hepatocellular, blood, kidney, mouth, prostate, liver, lung, skin, endocrine or testicular cancer.

Also included in the invention are kits for evaluating the efficacy of a cancer treatment in a mammal, comprising one or more reagents for detecting a level of Pin-1 in a test sample obtained from the mammal.

The invention described herein provides methods of detecting abnormal cell growth such as oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, or liposarcoma abnormal cell growth. Advantages of the claimed invention include, for example, the rapid and sensitive nature of detection in a cost effective manner. The methods of the invention can readily detect various stages of aggressive and/or metastasis of abnormal cell growth, e.g., breast or prostate cancer, thereby indicating an appropriate treatment method the progress of which can be monitored by the methods described in the invention.

The invention also provides a method for facilitating the diagnosis of a state associated with abnormal cell growth in a subject, comprising detecting the level of a Pin1 marker in a sample from the subject as an indication of whether the subject has a state associated with abnormal cell growth, thereby facilitating the diagnosis of the subject. The invention further provides a method for facilitating the diagnosis of cancer in a subject, comprising detecting the level of a Pin1 marker in a sample from the subject as an indication of whether the subject has cancer, thereby facilitating the diagnosis of the subject. In related embodiments, the subject is receiving, or has received, therapy for a state associated with abnormal cell growth and the diagnosis is used to evaluate the subject's response to the therapy. In yet another related embodiment, the subject is involved in a therapy agent clinical trial and the diagnosis is used to evaluate the effectiveness of an agent of the clinical trial.

The invention further provides a method for treating a subject wherein a Pin1 inhibitor is used in combination with radiation therapy.

Another aspect of the invention provides a method of treating a subject for a state associated with abnormal cell growth, comprising administering a Pin1 modulator to the subject such that the state associated with abnormal cell growth is treated. The invention further provides a method of treating a subject for cancer, comprising administering a Pin1 modulator to the subject such that the cancer is treated.

The invention described herein provides a packaged kit for carrying out a method of the invention, wherein the kit comprises at least one reagent for assaying levels of Pin1 in a sample from a subject, and instructions for using the at least one reagent to assay levels of Pin1 in a sample from a subject for the described method. The invention described herein further provides packaged kit for carrying out a method of the invention, wherein the kit comprises at least one Pin1 modulator, and instructions for using the Pin1 modulator in the described method.

The invention described herein also provides a pharmacogenomics method to determine which Pin1 inhibitor a given patient or cancer type will respond to most favorably.

The advantages of the invention are that this invention provides, to date, the best method to determine whether cancer will metastasize for breast, prostate and lung cancer. Further, the classification of high risk or low risk for metastasis can be made with out the invasive surgery that is currently used. Thus, the invention can determine the aggressiveness of therapy necessary without subjecting an individual to major surgery.

### DESCRIPTION OF THE FIGURES

FIG. 1 depicts an assay of Pin1 protein levels in 10 normal (non-cancerous) breast tissues and various stages of 51 breast cancer samples. Expression of actin was used to normalize values, and Pin1 levels are compared as Pin1/actin ratios. “DCIS” indicates “ductal carcinoma in situ”.

FIG. 2 depicts a statistical comparison of the quantified levels of Pin1 and other markers in normal and cancerous breast tissues. Pin1 levels are considered positive in this study if the Pin1/actin ration is higher than mean plus three times standard deviation (Xₘₑₐₙ±3SD) of normal controls. The presence of CyclinD1 and HER2/neu were determined by immunoblotting. Estrogen receptor was defined as positive if its levels were >10 fmol/l, as determined by RIA. (+00000=number of cases examined, *=estrogen receptors in controls not determined, ¶=estrogen receptor determination for one patient not available).

FIG. 3 depicts the significance of the differences in Pin1 levels between various clinical and pathological categories as analyzed by the Kruskall-Wallace Test. (+=analysis done only in tumors; * differences are statistically significant when P≦0.05 and highly significant when P≦0.01.

FIG. 4 depicts a number of genes whose expression is modulated (up- or down-regulation) by Pin1 overexpression in breast cancer cells.

FIG. 5 depicts a representation of the cyclin D1 (CD1) pA3LUC basic reporter constructs (and AP-1 site mutant) which were used in Pin1 overexpressing Hela and MCF-7 cells (Pin1^{AS} are the cells which overexpress the antisense construct). The activity of the reporter luciferase was expressed in relative activity in control vector transfected cells, which is defined as 1.0. Similar results were obtained in at least 3 different experiments. All results are expressed as Xₘₑₐₙ±SD of independent duplicate cultures.

FIG. 6 depicts further cyclin D1 promoter activation experiments transfected Hela cells. Pin1 is shown to cooperate with Ha-Ras in enhancing the c-Jun activity towards the cyclin D1 promoter.

Panel “a” shows a cotransfection experiment whereby Pin1 and Ha-Ras cooperate to increase the activity of c-Jun as a function of increasing amounts of transfected Pin1.

In this experiment, HeLa cells were cotransfected with vector, c-Jun or c-Jun +H-Ras, and different amounts of Pin1 expression vector for 24 hr and then subjected to the luciferase assay. The −964 cyclin D1-luciferase was used promoter as a reporter gene.

Panel “b” shows increasing or diminishing c-Jun activity by up- or down-regulation of Pin1. HeLa cells were cotransfected with different constructs, as indicated, and then subjected to the luciferase assay. Note, two different concentrations of Pin1^{AS} DNA (0.1 and 0.5 μg) were used, with a stronger inhibitory effect when more DNA was used.

Panel “c” shows abrogation of the ability of Pin1 to increase the c-Jun activity by mutation of the phosphorylation sites of c-jun (S63/73). Cells were co-transfected with Pin1, Ha-Ras, various amounts of c-Jun or c-Jun mutant S63/73A construct, as well as the −964 cyclin D1 luciferase reporter gene and then subjected to the luciferase assay.

Panel “d” shows inhibition of the ability of Pin1 to increase the c-Jun activity by dominant-negative Ras (DN-Ras). Cells were co-transfected with c-Jun or c-Jun +Pin1 and increasing amounts of DN-Ras, as well as the −964 cyclin D1 luciferase reporter gene, and then subjected to the luciferase assay.

Panel “e” shows abrogation of the ability of Pin1 to enhance c-Jun activity by inactivating (mutating) the Pin1 PPIase activity. Cells transfected with −964 cyclin D1 luciferase reporter gene were co-transfected with control vector, c-Jun, or c-Jun+Ha-Ras and Pin1 or its PPIase-negative mutant Pin1^{R68,69A} and then subjected to a luciferase assay. Pin1^{R68,69A} fails to isomerize phosphorylated S/T-P bonds.

Panel “f” shows abrogation of the ability of Pin1 to increase the c-Jun activity by inactivating (mutating) the Pin1 phosphoprotein-binding activity. Cells transfected with −964 cyclin D1 luciferase reporter gene were co-transfected with vectors, c-Jun, or c-Jun +Ha-Ras and GFP-Pin1 or one of its WW domain mutants GFP-Pin1^{W34A} or GFP-Pin1 ^{S16E}, then subjected to luciferase assay. Neither GFP-Pin1^{W34A} nor GFP-Pin1^{S16E} could bind phosphoproteins (data not shown). Note, GFP fusion proteins were used because these WW domain Pin1 mutants were not stable in cells, but when expressed as GFP fusion proteins, they were stable, although at reduced levels (data not shown). Although the absolute maximal luciferase activity was not as high as other experiments, which is likely due to lower levels of GFP fusion proteins being expressed, the overall trends were same.

FIG. 7 shows the correlation between Pin1 expression and Gleason sum based on 42 specimens of human prostate carcinomas with Gleason scores of 4-10. Each symbol represents a specimen from a different individual.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention relates to the discovery that the levels of Pin1 are elevated in cells undergoing abnormal cell growth (e.g., oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, and liposarcoma abnormal cell growth). The invention further relates to the discovery that the levels of Pin1 increase as a collection of cells undergoing abnormal cell growth, e.g., a tumor, become more aggressive, proliferative or metastasize. Thus, elevated levels of Pin1 are indicative of a tumor and are used as a tumor marker.

Pin1 is dramatically overexpressed in human cancer samples and the levels of Pin1 are correlated with the aggressiveness of tumors. Inhibition of Pin1 by various approaches, including the Pin1 inhibitor, Pin1 antisense polynucleotides, or genetic depletion, kills human and yeast dividing cells by inducing premature mitotic entry and apoptosis. Thus, upon phosphorylation, Pin1 latches onto phosphoproteins and twists the peptide bond next to the proline, which regulates the function of phosphoproteins and participates in controlling the timing of mitotic progression. This new regulatory mechanism not only will help the cell orchestrate the organized set of the mitotic events, but also is a novel and attractive target for drug development. Our studies also indicate that detection of Pin1 protein levels may be a novel universal tumor marker for identifying tumor cells and monitoring their aggressiveness and their response to cancer treatment, such as surgical, drug (e.g., chemotherapeutics) or radiation treatment.

In cells with cancer Pin1 is underexpressed in tissues that normally have a high level of Pin1 present (e.g., kidney and testis). In these cases an abnormally low level of Pin1 can be used as a marker that subjects have cancer in these tissues.

Uses and Methods of the Invention

The Pin1 markers (e.g., Pin1 nucleic acid molecules, Pin1 proteins, Pin1 protein homologues, and/or Pin1 antibodies) described herein can be used in one or more methods which relate to Pin1-associated disorders, including: a) screening assays ; b) predictive medicine (e.g., diagnostic assays, prognostic assays, monitoring clinical trials, and pharmacogenetics); and c) methods of treatment (e.g., therapeutic and prophylactic).

“Subject” includes living organisms, e.g., prokaryotes and eukaryotes. Examples of subjects include mammals, e.g., humans, dogs, cows, horses, kangaroos, pigs, sheep, goats, cats, mice, rabbits, rats, and transgenic non-human animals. Most preferably the subject is a human.

As used herein, the term “Pin1-associated disorder” includes a disorder or a state (e.g., a disease state) which is associated with abnormal cell growth, abnormal cell proliferation, or aberrant levels of Pin1 marker. Pin1-associated disorders include cancers, malignancies, tumors, and proliferative arthritic conditions. Pin1-associated disorders further include disorders which are not specific to a given tissue or cell type (e.g., a Pin1-associated disorder may present in a variety of tissues or cell types).

As used herein, the term “abnormal cell growth” is intended to include cell growth which is undesirable or inappropriate. Abnormal cell growth also includes proliferation which is undesirable or inappropriate (e.g., unregulated cell proliferation or undesirably rapid cell proliferation). Abnormal cell growth can be benign and result in benign masses of tissue or cells, or benign tumors. Many art-recognized conditions are associated with such benign masses or benign tumors including diabetic retinopathy, retrolental fibrioplasia, neovascular glaucoma, psoriasis, angiofibromas, rheumatoid arthritis, hemangiomas, and Karposi's sarcoma. Abnormal cell growth can also be malignant and result in malignancies, malignant masses of tissue or cells, or malignant tumors. Many art-recognized conditions and disorders are associated with malignancies, malignant masses, and malignant tumors including cancer and carcinoma.

As used herein, the term “tumor” is intended to encompass both in vitro and in vivo tumors that form in any organ of the body. Tumors may be associated with benign abnormal cell growth (e.g., benign tumors) or malignant cell growth (e.g., malignant tumors). The tumors which are described herein are sensitive to the Pin1 inhibitors of the present invention. Examples of the types of tumors intended to be encompassed by the present invention include those tumors casused by oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, and liposarcoma abnormal cell growth.

“Cancer” includes a malignant neoplasm characterized by deregulated or uncontrolled cell growth. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The histological features of cancer are summarized by the term “anaplasia.”Malignant neoplasms often contain numerous mitotic cells. These cells are typically abnormal. Such mitotic aberrations account for some of the karyotypic abnormalities found in most cancers. Bizarre multinucleated cells are also seen in some cancers, especially those which are highly anaplastic. “Dyplasia” refers to a pre-malignant state in which a tissue demonstrates histologic and cytologic features intermediate between normal and anaplastic. Dysplasia is often reversible.

“Anaplasia” refers to the histological features of cancer. These features include derangement of the normal tissue architecture, the crowding of cells, lack of cellular orientation termed dyspolarity, cellular heterogeneity in size and shape termed “pleomorphism.” The cytologic features of anaplasia include an increased nuclear-cytoplasmic ratio (the nuclear-cytoplasmic ratio can be over 50% for maligant cells), nuclear pleomorphism, clumping of the nuclear chromatin along the nuclear membrane, increased staining of the nuclear chromatin, simplified endoplasmic reticulum, increased free ribosomes, pleomorphism of mitochondria, decrease in size and number of organelles, enlarged and increased numbers of nucleoli, and sometimes the presence of intermediate filaments.

“Neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli which evoked the change. Neoplasms may show a partial or complete lack of structural organization and functional coordination with the normal tissue, and usually form a distinct mass of tissue.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated.

By assessing the histologic and other features of a neoplasm, it can be determined whether the neoplasm is benign or malignant. Invasion and metastasis (the spread of the neoplasm to distant sites) are definitive attributes of malignancy. Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. By contrast, malignant tumor generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue may be converted to a fibrous connective tissue capsule allowing for easy surgical remove of benign tumors. By contrast, malignant tumors are locally invasive and grow into the adjacent tissues usually giving rise to irregular margins that are not encapsulated making it necessary to remove a wide margin of normal tissue for the surgical removal of malignant tumors. Benign neoplasms tends to grow more slowly than malignant tumors. Benign neoplasms also tend to be less autonomous than malignant tumors. Benign neoplasms tend to closely histologically resemble the tissue from which they originated. More high differentiated cancers, cancers that resemble the tissue from which they originated, tend to have a better prognosis than poorly differentiated cancers. Malignant tumors are more likely than benign tumors to have an aberrant function (i.e. the secretion of abnormal or excessive quantities of hormones).

As used herein, the term “Pin1 marker” refers to a marker which is capable of being indicative of Pin1 levels in a sample of the invention. Pin1 markers include nucleic acid molecules (e.g., mRNA, DNA) which corresponds to some or all of a Pin1 gene, peptide sequences (e.g., amino acid sequences) which correspond to some or all of a Pin1 protein, peptide sequences which are homologous to Pin1 peptide sequences, antibodies to Pin1 protein, substrates of Pin1 protein, binding partners of Pin1 protein, and activity of Pin1.

The isolated nucleic acid molecules of Pin1 can be used, for example, to detect Pin1 mRNA (e.g., Pin1 nucleic acid marker in a biological sample) or a genetic alteration in a Pin1 gene. Moreover, the anti-Pin1 antibodies of the invention can be used to detect levels of Pin1 in a biological sample.

A. Screening Assays for Modulators and/or Inhibitors:

One major goal in cancer treatment has been to prevent the unregulated cell proliferation and, even better, to specifically kill dividing cancer cells. Interestingly, mitotic checkpoint controls have been identified as key targets for anticancer therapeutic procedures for two major reasons. First, since mitosis is a tightly regulated and orderly process, anticancer drugs that target at mitotic checkpoint controls can kill cells, often by inducing mitotic arrest followed by apoptosis. This is in contrast to those anticancer drugs that target other phase of the cell cycle, which just stop cells from continuous growing, but do not kill them. One of the best examples is the microtubule modifying agents, such as Oncovin and Taxols, which have been proven to be powerful drugs in treating various tumors (Piccart and Di Leo (1997) Semin Oncol 24:S10-27- S10-33). Second, abrogation of G2/M checkpoint have been shown to improve radiation therapy (Meyn (1997) Oncology 11:349-56 (see also discussion on pages 356, 361 and 365); Muscbel et al. (1997) Vitam Horm 53:1-25). Since effective radiation therapy has been shown to induces cell cycle arrest in G2 and M, and subsequent apoptosis, drugs that disrupt mitotic checkpoints would have a cooperative effect with irradiation in killing cancer cells. For at least the following reasons, Pin1 is be a potential novel drug target.

Pin1 is overexpressed in a variety of human cancer samples, including, but not limited to breast, uterus, ovarian, brain, endometrium, cervical, colon, esophagus, hepatocellular, kidney, mouth, prostate, liver, lung, skin, endocrine and testicularand its levels are correlated with the nuclear grade of tumors, as described above. These results suggest that Pin1 inhibitors are likely to have more selectivity to kill cancer cells.

B. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for measuring levels of Pin1 marker, as well as Pin1 activity, in the context of a biological sample to thereby determine whether an individual is afflicted with a disease or disorder, or is at risk of developing a disorder, associated with aberrant Pin1 expression or activity (e.g., abnormal or indignant cell growth, tumors, cancer). The invention also provides for prognostic (or predictive) assays for determining whether an individual is at risk of developing a disorder associated with a Pin1 marker. The invention further provides for prognostic (or predictive) assays for determining the stage of a Pin1-associated disorder.

As used herein, the term “stage” includes the degree of progression of a disease. Examples of Pin1-associated disorders which may have stages assigned to them include cancers, malignancies, abnormal cell growth, and tumors. Considerations for assigning stages to such disorders include level of metatsases (if metastatic at all) of a cancer or malignancy, and level of aggressiveness of a cancer or malignancy. Other generally accepted criteria for assigning stages to such disorders are well known to one skilled in the art.

Another aspect of the invention pertains to monitoring the effectiveness of agents (e.g., drugs, compounds, anti-cancer agents) on the expression or activity of Pin1 in clinical trials.

These and other agents are described in further detail in the following sections.

1. Diagnostic Assays

An exemplary method for detecting the presence or absence of Pin1 protein or nucleic acid in a biological sample involves obtaining a biological sample from a test subject and contacting the biological sample with a compound or an agent capable of detecting Pin1 protein or nucleic acid (e.g., mRNA, genomic DNA) that encodes Pin1 protein such that the presence of Pin1 protein or nucleic acid is detected in the biological sample. A preferred agent for detecting Pin1 mRNA or genomic DNA is a labeled nucleic acid probe capable of hybridizing to Pin1 mRNA or DNA. The nucleic acid probe can be, for example, a Pin1 nucleic acid or a corresponding nucleic acid such as an oligonucleotide of at least 15, 30, 50, 100, 250 or 500 nucleotides in length which is capable of specifically hybridizing under stringent conditions to Pin1 mRNA or genomic DNA. Other suitable probes for use in the diagnostic assays of the invention are described herein.

This invention provides a method for measuring the aggressiveness of cancer in a subject, comprising: (a) obtaining a cancer tissue sample from the subject; (b) contacting the tissue sample with an antibody to Pin1 or a fragment thereof to form a complex between the antibody and Pin1; (c) determining the amount of binding of the antibody to the tissue sample; and (d) comparing the amount of antibody bound to the tissue sample to a predetermined base level to measure the aggressiveness of the cancer, wherein increased amounts of the antibody bound to the tissue sample are diagnostic of a more aggressive cancer.

This invention further provides a method for identifying cancer likely to metastasize in a subject, comprising: (a) obtaining a cancer tissue sample from the subject; (b) contacting the tissue sample with an antibody to Pin1 to form a complex between the antibody and Pin1; (c) determining the amount of binding of the antibody to the tissue sample; and (d) comparing the amount of antibody bound to the tissue sample to a predetermined base level to measure the likelihood of the cancer to metastasize, wherein increased amounts of the antibody bound to the tissue sample are diagnostic of a cancer likely to metastasize.

In addition, this invention provides a method for diagnosing cancer in a subject, comprising: (a) obtaining a tissue sample from the subject; (b) contacting the tissue sample with an attached antibody to Pin1 to form a Pin1-antibody complex, wherein the attached antibody is attached to a solid phase; (c) contacting the Pin1-antibody complex with a probe antibody, wherein the probe antibody binds to a second site on Pin1; and (d) determining the amount of binding of the probe antibody to the tissue sample.

In the above methods, the amount of the complex between the antibody and Pin1 is determined by the intensity of the signal emitted by the labeled antibody or by the number cells in the tissue sample bound to the labeled antibody.

In preferred embodiments of the above diagnostic and prognostic methods, the abnormal cell growth or cancer is leukemia, prostate cancer, or breast cancer.

The above diagnostic and prognostic methods may be used in combination with other above diagnostic and prognostic methods. For example, the above methods may used on a subject or mammal that was identified by a blood test as possibly having leukemia or that was identified by a bone marrow test as possibly having leukemia. Similarly, the above methods may used on a subject or mammal that was identified as having stage I or II chronic lymphocytic leukemia under the Rai staging system. In addition, the above methods may used on a subject or mammal that was identified by mammography or breast ultrasound as having a breast abnormality. The above methods may used on a subject or mammal that was identified as having breast cancer tissue that is in stage III or which has a histological grade of 3 under the Scarff-Bloom-Richardson system.

Antibodies

“Antibody” includes immunoglobulin molecules and immunologically active determinants of immunoglobulin molecules, i.e., molecules that contain an antigen binding site which specifically binds (immunoreacts with) an antigen. Antibody includes polyclonal antibodies, monoclonal antibodies, whole immunoglobulins, and antigen binding fragments of the immunoglobulins.

A antibody fragments are obtained using conventional techniques well-known to those with skill in the art, and the fragments are screened for utility in the same manner as are intact antibodies. The term “antibody” is further intended to include bispecific and chimeric molecules having at least one antigen binding determinant derived from an antibody molecule.

In the diagnostic and prognostic assays of the invention, the antibody can be a polyclonal antibody or a monoclonal antibody and in a preferred embodiment is a labeled antibody.

Polyclonal antibodies are produced by immunizing animals, usually a mammal, by multiple subcutaneous or intraperitoneal injections of an immunogen (antigen) and an adjuvant as appropriate. As an illustrative embodiment, animals are typically immunized against a protein, peptide or derivative by combining about 1 μg to 1 mg of protein capable of eliciting an immune response, along with an enhancing carrier preparation, such as Freund's complete adjuvant, or an aggregating agent such as alum, and injecting the composition intradermally at multiple sites. Animals are later boosted with at least one subsequent administration of a lower amount, as ⅕ to 1/10 the original amount of immunogen in Freund's complete adjuvant (or other suitable adjuvant) by subcutaneous injection at multiple sites. Animals are subsequently bled, serum assayed to determine the specific antibody titer, and the animals are again boosted and assayed until the titer of antibody no longer increases (i.e., plateaus).

“Monoclonal antibody” or “monoclonal antibody composition” as used herein refers to a preparation of antibody molecules of single molecular composition. A monoclonal antibody composition displays a single binding specificity and affinity for a particular epitope. Monoclonal antibodies can be prepared using a technique which provides for the production of antibody molecules by continuous growth of cells in culture. These include but are not limited to the hybridoma technique originally described by Kohler and Milstein (1975, Nature 256:495-497; see also Brown et al. 1981 J. Immunol 127:539-46; Brown et al., 1980, J Biol Chem 255:4980-83; Yeh et al., 1976, PNAS 76:2927-31; and Yeh et al., 1982, Int. J. Cancer 29:269-75) and the more recent human B cell hybridoma technique (Kozbor et al., 1983, Immunol Today 4:72), EBV-hybridoma technique (Cole et al., 1985, Monoclonal Antibodies and Cancer Therapy, Alan R. Liss, Inc., pp. 77-96), and trioma techniques.

The fusion-product cells, which include the desired hybridomas, are cultured in selective medium such as HAT medium, designed to eliminate unfused parental myeloma or lymphocyte or spleen cells. Hybridoma cells are selected and are grown under limiting dilution conditions to obtain isolated clones. The supernatants of each clonal hybridoma is screened for production of antibody of desired specificity and affinity, e.g., by immunoassay techniques to determine the desired antigen such as that used for immunization. Monoclonal antibody is isolated from cultures of producing cells by conventional methods, such as ammonium sulfate precipitation, ion exchange chromatography, and affinity chromatography (Zola et al., Monoclonal Hybridoma Antibodies: Techniques And Applications, Hurell (ed.), pp. 51-52, CRC Press, 1982). Hybridomas produced according to these methods can be propagated in culture in vitro or in vivo (in ascites fluid) using techniques well known to those with skill in the art.

“Labeled antibody” as used herein includes antibodies that are labeled by a detectable means and includes enzymatically, radioactively, fluorescently, chemiluminescently, and/or bioluminescently labeled antibodies.

One of the ways in which an antibody can be detectably labeled is by linking the same to an enzyme. This enzyme, in turn, when later exposed to its substrate, will react with the substrate in such a manner as to produce a chemical moiety which can be detected, for example, by spectrophotometric, fluorometric or by visual means. Enzymes which can be used to detectably label the Pin1-specific antibody include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection may be accomplished using any of a variety of immunoassays. For example, by radioactively labeling an antibody, it is possible to detect the antibody through the use of radioimmune assays. A description of a radioimmune assay (RIA) may be found in Laboratory Techniques and Biochemistry in Molecular Biology, by Work, T. S., et al., North Holland Publishing Company, NY (1978), with particular reference to the chapter entitled “An Introduction to Radioimmune Assay and Related Techniques” by Chard, T.

The radioactive isotope can be detected by such means as the use of a gamma counter or a scintillation counter or by audioradiography. Isotopes which are particularly useful for the purpose of the present invention are: ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthamide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

Likewise, a bioluminescent compound may be used to label an antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Additionally, antibodies directed toward a protein of interest can be connected to magnetic beads and used to enrich a population. Immunomagnetic selection has been used previously for this purpose and examples of this method can be found, for example, at U.S. Pat. No. 5,646,001; Ree et al. (2002) Int. J. Cancer 97:28-33; Molnar et al. (2001) Clin. Cancer Research 7:4080-4085; and Kasimir-Bauer et al. (2001) Breast Cancer Res. Treat. 69:123-32. An antibody, either polyclonal or monoclonal, that is specific for a cell surface protein on a cell of interest can be attacthed to a magnetic substrate thereby allowing selection of only those cells that express the surface protein of interest. The selected cells can then be lysed and the cellular contents assayed for the presence of Pin1.

In the diagnostic and prognostic assays of the invention, the amount of binding of the antibody to the tissue sample can be determined by the intensity of the signal emitted by the labeled antibody and/or by the number cells in the tissue sample bound to the labeled antibody.

Immunoassays

The amount of an antigen (i.e. Pin1) in a tissue sample may be determined by a radioimmunoassay, an immunoradiometric assay, and/or an enzyme immunoassay.

“Radioimmunoassay” is a technique for detecting and measuring the concentration of an antigen using a labeled (i.e. radioactively labeled) form of the antigen. Examples of radioactive labels for antigens include ³H, ¹⁴C, and ¹²⁵I. The concentration of antigen (i.e. Pin1 ) in a sample (i.e. tissue sample) is measured by having the antigen in the sample compete with a labeled (i.e. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the test sample can be determined.

A “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” The “Enzyme-Linked Immunosorbent Assay (ELISA)” is a technique for detecting and measuring the concentration of an antigen using a labeled (i.e. enzyme linked) form of the antibody.

In a “sandwich ELISA”, an antibody (i.e. to Pin1) is linked to a solid phase (i.e. a microtiter plate) and exposed to a test sample containing antigen (i.e. Pin1). The solid phase is then washed to remove unbound antigen. A labeled (i.e. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be assayed for.

In a “competitive ELISA”, antibody is incubated with a sample containing antigen (i.e. Pin1). The antigen-antibody mixture is then contacted with an antigen-coated solid phase (i.e. a microtiter plate). The more antigen present in the sample, the less free antibody that will be available to bind to the solid phase. A labeled (i.e. enzyme linked) secondary antibody is then added to the solid phase to determine the amount of primary antibody bound to the solid phase.

A preferred agent for detecting Pin1 marker is an antibody capable of binding to Pin1 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

With respect to antibody-based detection techniques, one of skill in the art can raise anti-Pin1 antibodies against an appropriate immunogen, such as isolated and/or recombinant Pin1 or a portion or fragment thereof (including synthetic molecules, such as synthetic peptides) using no more than routine experimentation. Synthetic peptides can be designed and used to immunize animals, such as rabbits and mice, for antibody production. The nucleic and amino acid sequence of Pin1 is known (Hunter et al., WO 97/17986 (1997); Hunter et al., U.S. Pat. Nos. 5,952,467 and 5,972,697, the teachings of all of which are hereby incorporated by reference in their entirety) and can be used to design nucleic acid constructs for producing proteins for immunization or in nucleic acid detection methods or for the synthesis of peptides for immunization. Conditions for incubating an antibody with a test sample can vary depending upon the tissue or cellular type. Incubation conditions can depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays) can readily be adapted to employ the antibodies of the present invention. Examples of such assays can be found in Chard, “An Introduction to Radioimmunoassay and Related Techniques,” Elsevier Science Publishers, Amsterdam, The Netherlands (1986); Bullock et al., “Techniques in Immunocytochemistry,” Academic Press, Orlando, Fla. Vol. 1 (1982), Vol. 2 (1983), Vol. 3 (1985); Tijssen, “Practice and Theory of enzyme Immunoassays: Laboratory Techniques in Biochemistry and Molecular Biology,” Elsevier Science Publishers, Amsterdam, The Netherlands (1985).

As used herein, the terms “sample,” “test sample,” “tissue sample,” and “biological sample” include samples obtained from a mammal or a subject containing Pin1 which can be used within the methods described herein, e.g., tissues, cells and biological fluids isolated from a subject, as well as tissues, cells and fluids present within a subject. “Tissue samples” include solid and liquid tissue samples. Examples of solid tissue samples include samples taken from the rectum, central nervous system, bone, breast tissue, renal tissue, the uterine cervix, the endometrium, the head/neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of “liquid tissue samples” or “body fluid samples” include samples taken from the blood, serum, semen, prostate fluid, seminal fluid, urine, saliva, sputum, phlegm, pus, mucus, bone marrow, lymph, ascites and tears. For amplifying Pin1 RNA, the preferred tissue sample is a peripheral venous blood sample.

Accordingly, the detection method of the invention can be used to detect Pin1 mRNA, protein, or genomic DNA in a biological sample in vitro as well as in vivo. For example, in vitro techniques for detection of Pin1 mRNA include Northern hybridizations and in situ hybridizations. In vitro techniques for detection of Pin1 protein include enzyme linked immunosorbent assays (ELISAs), Western blots, immunoprecipitations and immunofluorescence. In vitro techniques for detection of Pin1 genomic DNA include Southern hybridizations. Furthermore, in vivo techniques for detection of Pin1 protein include introducing into a subject a labeled anti-Pin1 antibody. For example, the antibody can be labeled with a radioactive marker whose presence and location in a subject can be detected by standard imaging techniques.

In another embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting Pin1 marker such that the presence of Pin1 marker is detected in the biological sample, and comparing the presence of Pin1 marker in the control sample with the presence of Pin1 marker in the test sample.

The immunological assay test samples of the present invention may include cells, protein or membrane extracts of cells, blood or biological fluids such as ascites fluid or brain fluid (e.g., cerebrospinal fluid). The test sample used in the above-described method is based on the assay format, nature of the detection method and the tissues, cells or extracts used as the sample to be assayed. Methods for preparing protein extracts or membrane extracts of cells are well known in the art and can be readily be adapted in order to obtain a sample which is capable with the system utilized. The invention also encompasses kits for detecting the presence of Pin1 in a biological sample. For example, the kit can comprise a labeled compound or agent capable of detecting Pin1 protein or mRNA in a biological sample; means for determining the amount of Pin1 in the sample; and means for comparing the amount of Pin1 in the sample with a standard. The compound or agent can be packaged in a suitable container. The kit can further comprise instructions for using the kit to detect Pin1 protein or nucleic acid.

A compartmentalized kit can include any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe, primers or antibodies used in the assay, containers which contain wash reagents (such as phosphate buffered saline, Tris-buffers, and the like), and containers which contain the reagents used to detect the hybridized probe, bound antibody, amplified product, or the like.

The kits are used to detect and distinguish normal cells from cells undergoing abnormal cell growth. Additionally, or alternatively, the kits are used to distinguish between aggressive or various stages of an abnormal cell growth (e.g., breast, prostate, liver, lung, kidney, digestive track, ovarian, testicular, skin cancer) or to distinguish between benign or malignant forms of abnormal cell growth in tumors. It is also envisioned that the kits and methods of the invention can be used to define the need for treatment of abnormal cell growths, such as surgical interventions, types of chemotherapeutic drugs or radiation treatments.

The kits and methods of the invention are used to detect metastasis of abnormally cell growths. A “metastasis” is the spread of an abnormal cell growth from one part of the body (e.g., breast tissue, prostate gland, uterus, skin, testes, ovary) to another part of the body (e.g., breast, prostate, uterus, brain, skin, testes, ovary, lymph nodes). The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler, et al., Adv. Cancer Res. 28, 149-250 (1978), Liotta, et al., Cancer Treatment Res. 40, 223-238 (1988), Nicolson, Biochim. Biophy. Acta 948, 175-224 (1988) and Zetter, N. Eng. J. Med. 322, 605-612 (1990)). Increased malignant cell motility has been associated with enhanced metastatic potential in animal as well as human tumors (Hosaka, et al., Gann 69, 273-276 (1978) and Haemmerlin, et al., Int. J. Cancer 27, 603-610 (1981)).

“Invasive” or “aggressive” as used herein with respect to cancer refers to the proclivity of a tumor for expanding beyond its boundaries into adjacent tissue, or to the characteristic of the tumor with respect to metastasis (Darnell, J. (1990), Molecular Cell Biology, Third Ed., W. H.Freeman, NY). Invasive cancer can be contrasted with organ-confined cancer. For example, a basal cell carcinoma of the skin is a non-invasive or minimally invasive tumor, confined to the site of the primary tumor and expanding in size, but not metastasizing. In contrast, the cancer melanoma is highly invasive of adjacent and distal tissues. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located.

One skilled in the art will readily recognize that the nucleic acid probes described in the present invention can readily be incorporated into one of the established kit formats which are well known in the art.

In the embodiments of the invention described herein, well known biomolecular methods such as northern blot analysis, RNase protection assays, southern blot analysis, western blot analysis, in situ hybridization, immunocytohemical procedures of tissue sections or cellular spreads, and nucleic acid amplification reactions (e.g., polymerase chain reactions) may be used interchangeably. One of skill in the art would be capable of performing these well- established protocols for the methods of the invention. (See, for example, Ausubel, et al., “Current Protocols in Moleculer Biology,” John Wiley & Sons, NY, N.Y. (1999)).

2. Prognostic Assays

The diagnostic methods described herein can furthermore be utilized to identify subjects having or at risk of developing a disease or disorder associated with aberrant Pin1 expression or activity. For example, the assays described herein, such as the preceding diagnostic assays or the following assays, can be utilized to identify a subject having or at risk of developing a disorder associated with Pin1 marker (e.g., abnormal or malignant cell growth, tumors, cancer). Thus, the present invention provides a method for identifying a disease or disorder associated with aberrant Pin1 expression or activity in which a test sample is obtained from a subject and Pin1 protein or nucleic acid (e.g., mRNA, genomic DNA) is detected, wherein the presence of Pin1 protein or nucleic acid is diagnostic for a subject having or at risk of developing a Pin1-associated disorder.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant Pin1 expression or activity. Thus, the present invention provides methods for determining whether a subject can be effectively treated with an agent for a disorder associated with aberrant Pin1 expression or activity in which a test sample is obtained and Pin1 protein or nucleic acid expression or activity is detected (e.g., wherein the abundance of Pin1 protein or nucleic acid expression or activity is diagnostic for a subject that can be administered the agent to treat a disorder Pin1-associated disorder).

The methods of the invention can also be used to detect genetic alterations in a Pin1 gene, thereby determining if a subject with the altered gene is at risk for a disorder associated with the Pin1 gene. In preferred embodiments, the methods include detecting, in a sample of cells from the subject, the presence or absence of a genetic alteration characterized by at least one of an alteration affecting the integrity of a gene encoding a Pin1-protein, or the mis-expression of the Pin1 gene. For example, such genetic alterations can be detected by ascertaining the existence of at least one of 1) a deletion of one or more nucleotides from a Pin1 gene; 2) an addition of one or more nucleotides to a Pin1 gene; 3) a substitution of one or more nucleotides of a Pin1 gene, 4) a chromosomal rearrangement of a Pin1 gene; 5) an alteration in the level of a messenger RNA transcript of a Pin1 gene, 6) aberrant modification of a Pin1 gene, such as of the methylation pattern of the genomic DNA, 7) the presence of a non-wild type splicing pattern of a messenger RNA transcript of a Pin1 gene, 8) a non-wild type level of a Pin1-protein, 9) allelic loss of a Pin1 gene, and 10) inappropriate post-translational modification of a Pin1-protein. As described herein, there are a large number of assay techniques known in the art which can be used for detecting alterations in a Pin1 gene. A preferred biological sample is a tissue or serum sample isolated by conventional means from a subject, e.g., a cardiac tissue sample.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the Pin1-gene (see Abravaya et al. (1995) Nucleic Acids Res 0.23:675-682). This method can include the steps of collecting a sample from a patient, isolating nucleic acid (e.g., genomic, mRNA or both) from the sample, contacting the nucleic acid sample with one or more primers which specifically hybridize to a Pin1 gene under conditions such that hybridization and amplification of the Pin1-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a Pin1 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in Pin1 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in Pin1 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential ovelapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the Pin1 gene and detect mutations by comparing the sequence of the sample Pin1 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the Pin1 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type Pin1 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in Pin1 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a Pin1 sequence, e.g., a wild-type Pin1 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in Pin1 genes. For example, single strand conformation polymorphism (SSCP) may be used to detect differences in electrophoretic mobility between mutant and wild type nucleic acids (Orita et al. (1989) Proc Natl. Acad Sci USA: 86:2766, see also Cotton (1993) Mutat Res 285:125-144; and Hayashi (1992) Genet Anal Tech Appl 9:73-79). Single-stranded DNA fragments of sample and control Pin1 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner et al. (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a Pin1 gene.

Furthermore, any cell type or tissue in which Pin1 is expressed may be utilized in the prognostic assays described herein.

3. Monitoring of Effects During Clinical Trials

Monitoring the influence of agents (e.g., drugs or compounds) on the expression or activity of a Pin1 protein can be applied not only in basic drug screening, but also in clinical trials. For example, the effectiveness of an agent determined by a screening assay as described herein to increase Pin1 gene expression, protein levels, or upregulate Pin1 activity, can be monitored in clinical trials of subjects exhibiting decreased Pin1 gene expression, protein levels, or downregulated Pin1 activity. Alternatively, the effectiveness of an agent determined by a screening assay to decrease Pin1 gene expression, protein levels, or downregulate Pin1 activity, can be monitored in clinical trials of subjects exhibiting increased Pin1 gene expression, protein levels, or upregulated Pin1 activity. In such clinical trials, the expression or activity of a Pin1 gene, and preferably, other genes that have been implicated in a disorder can be used as a “read out” or markers of the phenotype of a particular cell.

For example, and not by way of limitation, genes, including Pin1, that are modulated in cells by treatment with an agent (e.g., compound, drug or small molecule) which modulates Pin1 activity (e.g., identified in a screening assay as described herein) can be identified. Thus, to study the effect of agents on a Pin1 associated disorder, for example, in a clinical trial, cells can be isolated and RNA prepared and analyzed for the levels of expression of Pin1 and other genes implicated in the Pin1 associated disorder, respectively. The levels of gene expression (i e., a gene expression pattern) can be quantified by Northern blot analysis or RT-PCR, as described herein, or alternatively by measuring the amount of protein produced, by one of the methods as described herein, or by measuring the levels of activity of Pin1 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression or activity of a Pin1 protein, mRNA, or genomic DNA in the pre-administration sample; (iii) obtaining one or more post-administration samples from the subject; (iv) detecting the level of expression or activity of the Pin1 protein, mRNA, or genomic DNA in the post-administration samples; (v) comparing the level of expression or activity of the Pin1 protein, mRNA, or genomic DNA in the pre-administration sample with the Pin1 protein, mRNA, or genomic DNA in the post administration sample or samples; and (vi) altering the administration of the agent to the subject accordingly.

C. Methods of Treatment:

The present invention provides for both prophylactic and therapeutic methods of treating a subject at risk of (or susceptible to) a disorder or having a disorder associated with aberrant Pin1 expression or activity (e.g., abnormal or malignant cell growth, tumors, cancer).

Also provided by this invention is a method for treating cancer in a subject comprising administering to a subject an effective amount of a combination of a Pin1 inhibitor and a hyperplastic inhibitory agent such that the cancer is treated.

In an embodiment of the above methods of treating abnormal cell growth or cancer, the treating includes inhibiting tumor growth and/or preventing the occurrence of tumor growth in the subject.

In an other embodiment of the above methods of treating abnormal cell growth or cancer, the treating includes a combination treatment in which a Pin1 inhibitor is administered to a subject in combination with radiation therapy.

In another embodiment of the above methods of treating abnormal cell growth or cancer, the abnormal cell growth or tumor growth or cancer is caused by overexpression of Pin1. In a preferred embodiment, the abnormal cell growth or tumor growth or cancer being treated is breast cancer, prostate cancer, or leukemia.

“Treatment”, as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the Pin1 molecules of the present invention or Pin1 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant Pin1 expression or activity, by administering to the subject a Pin1 or an agent which modulates Pin1 expression or at least one Pin1 activity. Subjects at risk for a disease which is caused or contributed to by aberrant Pin1 expression or activity can be identified by, for example, any of a combination of diagnostic or prognostic assays as described herein. Administration of a prophylactic agent can occur prior to the manifestation of symptoms characteristic of the Pin1 aberrancy, such that a disease or disorder is prevented or, alternatively, delayed in its progression. Depending on the type of Pin1 aberrancy, for example, a Pin1, Pin1 agonist or Pin1 antagonist agent can be used for treating the subject. The appropriate agent can be determined based on screening assays described herein.

2. Therapeutic Methods

Another aspect of the invention pertains to methods of modulating Pin1 expression or activity for therapeutic purposes. Accordingly, in an exemplary embodiment, the modulatory method of the invention involves contacting a cell with a Pin1 or agent that modulates one or more of the activities of Pin1 protein activity associated with the cell. An agent that modulates Pin1 protein activity can be an agent as described herein, such as a nucleic acid or a protein, a naturally-occurring target molecule of a Pin1 protein (e.g., a phosphoprotein), a Pin1 antibody, a Pin1 agonist or antagonist, a peptidomimetic of a Pin1 agonist or antagonist, or other small molecule. In one embodiment, the agent stimulates one or more Pin1 activities. Examples of such stimulatory agents include active Pin1 protein and a nucleic acid molecule encoding Pin1 that has been introduced into the cell. In another embodiment, the agent inhibits one or more Pin1 activites. Examples of such inhibitory agents include antisense Pin1 nucleic acid molecules, anti-Pin1 antibodies, and Pin1 inhibitors. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a Pin1 protein or nucleic acid molecule. In one embodiment, the method involves administering an agent (e.g., an agent identified by a screening assay described herein), or combination of agents that modulates (e.g., upregulates or downregulates) Pin1 expression or activity. In another embodiment, the method involves administering a Pin1 protein or nucleic acid molecule as therapy to compensate for reduced or aberrant Pin1 expression or activity.

Stimulation of Pin1 activity is desirable in situations in which Pin1 is abnormally downregulated and/or in which increased Pin1 activity is likely to have a beneficial effect. For example, stimulation of Pin1 activity is desirable in situations in which a Pin1 is downregulated and/or in which increased Pin1 activity is likely to have a beneficial effect. Likewise, inhibition of Pin1 activity is desirable in situations in which Pin1 is abnormally upregulated and/or in which decreased Pin1 activity is likely to have a beneficial effect.

The present invention further includes therapeutic methods which utilize a combination of therapeutic agents of the invention, as described herein, and further therapeutic agents which are known in the art. Specifically, a Pin1 modulator of the present invention can be used in combination with a second modulator or with a second “abnormal cell growth inhibitory agent” (ACI agent). The ACI agent can be any therapeutic agent which can be used to treat the selected Pin1-associated disorder and/or cancer. One skilled in the art would be able to select appropriate ACI agents for combination therapy with a Pin1 modulator. For example, an ACI agent may be a second Pin1 modulator, or it may be an art-recognized agent which does not modulate Pin1.

The terms “abnormal cell growth inhibitory agent” and “ACI agent” are used interchangeably herein and are intended to include agents that inhibit the growth of proliferating cells or tissue wherein the growth of such cells or tissues is undesirable. For example, the inhibition can be of the growth of malignant cells such as in neoplasms or benign cells such as in tissues where the growth is inappropriate. Examples of the types of agents which can be used include chemotherapeutic agents, radiation therapy treatments and associated radioactive compounds and methods, and immunotoxins.

The language “chemotherapeutic agent” is intended to include chemical reagents which inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases, tumors, and cancers.

The language “radiation therapy” is intended to include the application of a genetically and somatically safe level of x-rays, both localized and non-localized, to a subject to inhibit, reduce, or prevent symptoms or conditions associated with undesirable cell growth. The term x-rays is intended to include clinically acceptable radioactive elements and isotopes thereof, as well as the radioactive emissions therefrom. Examples of the types of emissions include alpha rays, beta rays including hard betas, high energy electrons, and gamma rays. Radiation therapy is well known in the art (see e.g., Fishbach, F., Laboratory Diagnostic Tests, 3rd Ed., Ch. 10: 581-644 (1988)), and is typically used to treat neoplastic diseases, tumors, and cancers.

The term “immunotoxins” includes immunotherapeutic agents which employ cytotoxic T cells and/or antibodies, e.g., monoclonal, polyclonal , phage antibodies, or fragments thereof, which are utilized in the selective destruction of undesirable rapidly proliferating cells. For example, immunotoxins can include antibody-toxin conjugates (e.g., Ab-ricin and Ab-diptheria toxin), antibody-radiolabels (e.g., Ab-I¹³⁵) and antibody activation of the complement at the tumor cell. The use of immunotoxins to inhibit, reduce, or prevent symptoms or conditions associated with neoplastic diseases are well known in the art (see e.g., Harlow, E. and Lane, D., Antibodies, (1988)).

The language “inhibiting undesirable cell growth” is intended to include the inhibition of undesirable or inappropriate cell growth. The inhibition is intended to include inhibition of proliferation including rapid proliferation. For example, the cell growth can result in benign masses or the inhibition of cell growth resulting in malignant tumors. Examples of benign conditions which result from inappropriate cell growth or angiogenesis are diabetic retinopathy, retrolental fibrioplasia, neovascular glaucoma, psoriasis, angiofibromas, rheumatoid arthritis, hemangiomas, Karposi's sarcoma, and other conditions or dysfunctions characterized by dysregulated endothelial cell division.

3. Pharmacogenomics

The Pin1 molecules of the present invention, as well as agents, or modulators which have a stimulatory or inhibitory effect on Pin1 activity (e.g., Pin1 gene expression) as identified by a screening assay described herein can be administered to individuals to treat (prophylactically or therapeutically) disorders (e.g, proliferative disorders such as cancer) associated with aberrant Pin1 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a Pin1 molecule or Pin1 modulator as well as tailoring the dosage and/or therapeutic regimen of treatment with a Pin1 molecule or Pin1 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11) :983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed, the “candidate gene approach”, can be utilized to identify genes that predict a drug response. According to this method, if a gene that encodes a drug target is known (e.g., a Pin1 protein or Pin1 receptor of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a Pin1 molecule or Pin1 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a Pin1 molecule or Pin1 modulator, such as a modulator identified by one of the exemplary screening assays described herein.

4. Use of Pin1 Molecules as Surrogate Markers

The Pin1 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the Pin1 molecules of the invention may be detected, and may be correlated with one or more biological states in vivo. For example, the Pin1 molecules of the invention may serve as surrogate markers for one or more disorders or disease states or for conditions leading up to disease states.

As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the causation of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35:258-264; and James (1994) AIDS Treatment News Archive 209.

The Pin1 marker molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a Pin1 marker) transcription or expression, the amplified marker may be in a quantity which is more readily detectable than the drug itself. Also, the marker may be more easily detected due to the nature of the marker itself; for example, using the methods described herein, anti-Pin1 antibodies may be employed in an immune-based detection system for a Pin1 protein marker, or Pin1-specific radiolabeled probes may be used to detect a Pin1 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90:229-238; Schentag (1999) Am. J. Health-Syst. Pharm. 56 Suppl. 3:S21-S24; and Nicolau (1999) Am. J. Health-Syst. Pharm. 56 Suppl. 3:S16-S20.

The Pin1 marker molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12):1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., Pin1 protein or RNA) for specific tumor markers in a subject, a drug or course of treatment may be selected that is optimized for the treatment of the specific tumor likely to be present in the subject. Similarly, the presence or absence of a specific sequence mutation in Pin1 DNA may correlate Pin1 drug response. The use of pharmacogenomic markers therefore permits the application of the most appropriate treatment for each subject without having to administer the therapy.

This invention is further illustrated by the following examples which should not be construed as limiting. The following examples show the use of Pin1 as a universal marker for abnormal cell growth, e.g., cancer and the involvement of Pin1 in tumorigenic pathways. The contents of all references, patents and published patent applications cited throughout this application, as well as the Figures, are incorporated herein by reference.

### EXAMPLES

### Example 1

### Pin1 is a Breast Tumor Marker

To determine whether Pin1 is overexpressed in human tumor samples, we examined the levels of Pin1 in human breast cancer samples using immunoblotting and immunohistochemical analysis with Pin1 antibodies, as described previously (Lu et al. (1999) Nature 399:784-788). Immunocytochemistry of sections of human breast tumors showed that Pin1 is indeed overexpressed in human breast tumor cells. Pin1 was detected both in the cytoplasm and in the nucleus, as well as in condensed chromosomes and mitotic spindles. Infiltrating carcinoma cells were strongly positive for Pin1 staining, while surrounding normal connective tissue, blood vessels, adipose, and stromal cells were only weakly positive. To ensure that these signals represent Pin1, a control immunostaining was performed whereby the Pin1-specific antibodies were first specifically depleted by pre-incubation with glutathione beads containing GST-Pin1. This depletion resulted in no detection of signal, demonstrating the specificity of the Pin1 antibodies used in the immunostaining. Furthermore, similar immunostaining in various breast tumor-derived cell lines, when compared with those in non-transformed mammary cell lines, showed significantly elevated expression of Pin1.

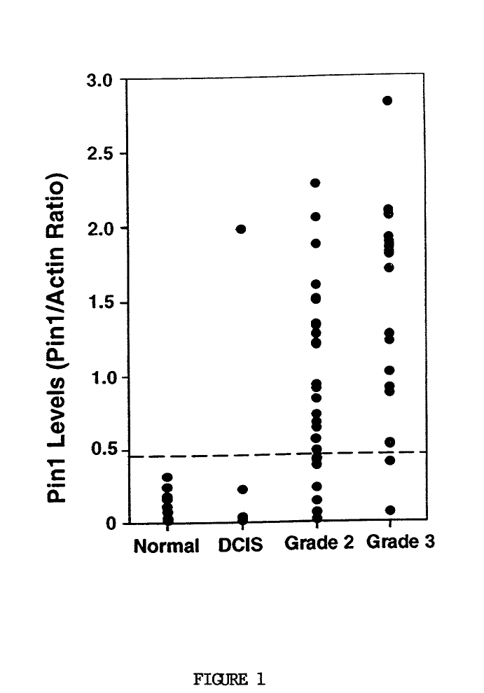

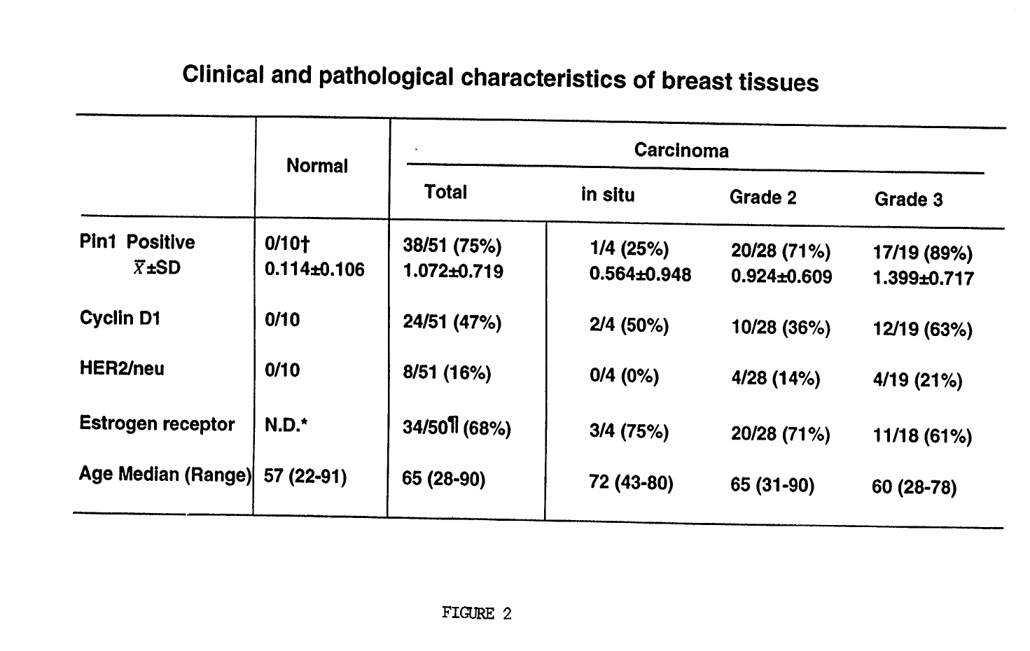

To confirm the immunostaining results and to establish a quantitative relationship between Pin1 expression and various known tumor markers, fresh normal and tumor breast tissues were ground in liquid nitrogen and lysates were directly subjected to immunoblotting analysis with various antibodies. Quantification of protein levels was carried out with “Imagequant” software, as described elsewhere (Lu et al. (1999) Nature 399:784-788). Using actin expression as a normalization control, Pin1 levels were compared as ratios of Pin1/actin expression. Using 10 non-cancerous breast tissue samples and 51 primary breast cancer tissue samples, we observed striking differences in levels of Pin1 protein between normal and neoplastic breast tissues. 71.4% of Grade II tumors and 89.5% of Grade III tumors overexpressed Pin1, wherein overexpression was defined as higher than mean plus three times standard deviation of the normal controls (FIGS. 1 and 2). Moreover, Pin1 levels positively correlated with the nuclear grade in invasive cancer, which is an important predictor of clinical aggressiveness of the tumors (Bloom-Richardson's classification; see, e.g., Bloom and Richardson, (1957) Br. J. Cancer, 11:359-377, and Bloom et al. (1962), Brit. Med. J 5299:213). Taken together, these results indicate that Pin1 is over expressed in the majority of breast cancer samples, with the highest expression levels in high-grade tumors.

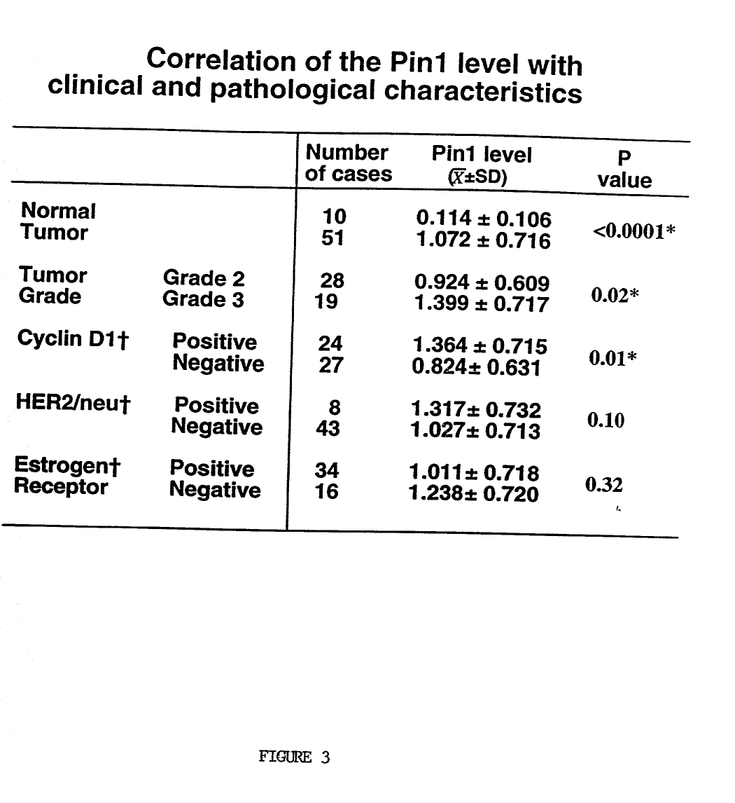

Pin1 levels were also compared to the levels of other known cancer markers (FIG. 3). It was observed that Pin1 levels did not correlate with either estrogen receptor or HER2/neu expression, but significantly correlated with cyclin D1 overexpression, as analyzed by the Kruskall-Wallis test (see, e.g., Glantz, S. A. (1997) Primer of Biostatistics, 4^{th} ed. McGraw Hill New York, pp346-348). As expected, cyclin D1 was overexpressed in about 50% of the patent samples (24 out of 51). Importantly, Pin1 was overexpressed in 20 out of 24 cyclin D1 overexpressing tumors. Moreover, the level of Pin1 in these tumors was about twice as high (on average) as in cyclin D1 negative tumors. The correlation between Pin1 and cyclin D1 expression indicate that overexpression of Pin1 can be correlated with expression of endogenous cyclin D1.

In order to test for a causative correlation between Pin1 and cyclin D1 expression a breast tumor cell line (MCF-7) was stably transfected such that Pin1 is expressed under the control of the tetracycline-regulated promoter. Although expression of actin was not affected in these cells, induction of Pin1 expression resulted in about a 2.5 fold increase in cyclin D1 protein levels in two independent cell lines, while cyclin D1 levels remained stable in uninduced cells. These results demonstrate that up-regulation of Pin1 causes overexpression of endogenous cyclin D1 in human breast cancer cell lines.

Further immunoblotting and quantification experiments revealed that levels of Pin1 protein and beta-catenin protein can be correlated in breast cancer cells. Beta-catenin is a gene which is known to be involved in certain tumorigenic pathways (see, e.g., Polakis, (2000) Genes Dev 14:1837-51, Behrens, (2000) N. Y Acad Sci 910:21-35; and Peifer and Polakis, (2000) Science 287:1606-9).

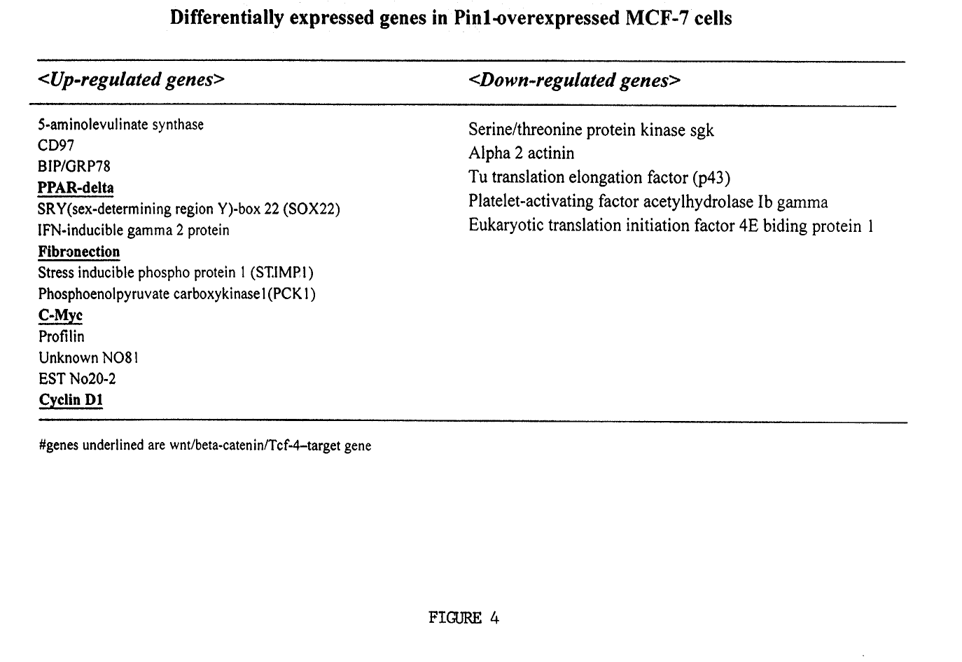

The expression of various other beta-catenin downstream target genes in Pin1-overexpressed MCF-7 cells was assessed using standard differential expression techniques (see, e.g., Ryo, et al (1998) Nucleic Acids Res 26:2586-92. The results are set forth in FIG. 4.

### Example 2

### Pin1 is a Colon Tumor Marker

To explore whether Pin1 is also overexpressed in colon tumors, we have examined the Pin1 levels in several human colon tumor samples using immunostaining and immunoblotting analyses (using the experimental methods set forth in Example 1). Pin1 was overexpressed in most samples examined, as compared with normal colon samples. These results indicate that Pin1 can act as marker for colon cancer.

### Example 3

### Pin1 as a Prostate Tumor Marker

To explore whether Pin1 is also overexpressed in prostate tumors, we have examined the Pin1 levels in several human prostate tumor samples using immunostaining and immunoblotting analyses (using the experimental methods set forth in Example 1). Pin1 was overexpressed in most samples examined, as compared with normal prostate samples. These results indicate that Pin1 can act as marker for prostate cancer.

### Example 4

### Pin1 is a Universal Marker of Proliferation

To further evaluate the potential of detecting Pin1 levels as a general marker for cell proliferation, the expression of Pin1 in an array of normal human tissues was assessed. A panel of 30 normal human tissues were stained with affinity-purified anti-Pin1 antibodies. Although very low levels of Pin1 were detected in non-epithelial cell types, such as different kinds of muscles, Pin1 was primarily detected at moderate levels in various types of epithelial cells, hemopoietic cells and at very high levels in germline cells of testis and ovary, especially in sperm. Specifically, it was observed that Pin1 expression in normal human tissues was associated with proliferative status. For example, cell proliferation primarily occurs at the base portion of clefts in colon and they stop proliferation when they move up along the cleft. In such areas, a gradient in the level of Pin1 signal was observed, e.g., Pin1 levels were much higher in the base portion than that in upper portion of clefts in colon. Similar phenomena were also observed in other tissues, such as the transitional epithelial cells of bladder. With the exception of testis, Pin1 levels in normal human tissues are much lower than those observed in human breast or prostate tumor samples. These results further indicate that detection of Pin1 levels can be used as a diagnostic marker for abnormal proliferation in an array of human tissues and diseases.

### Example 5

### Pin1 is Involved in Tumorigenic Pathways

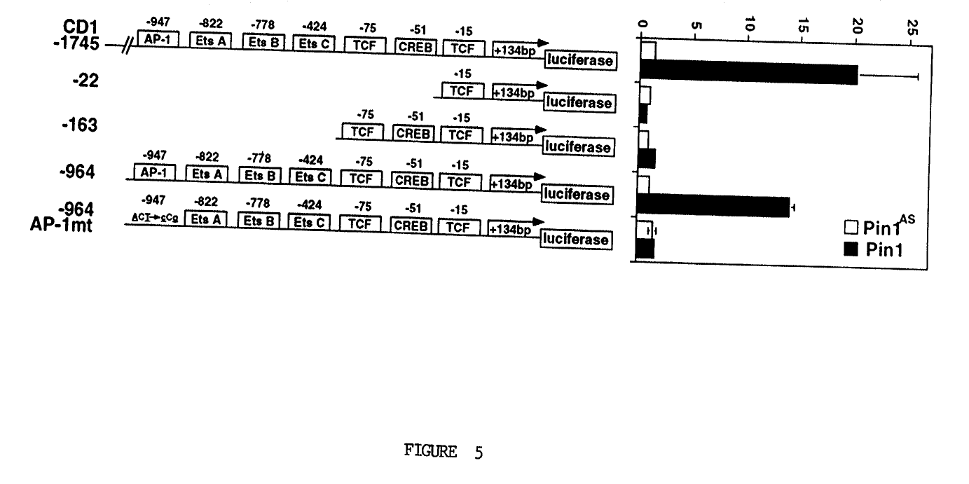

The role of Pin1 in the modulation of various known tumorigenic pathways, such as those associated with beta-catenin and cyclin D1, was investigated in more detail. Although cyclin D1 overexpression is found in ˜50% of breast cancer patients (Gillett, et al. (1994) Cancer Res 54:1812-1817, Bartkova, et al. (1994) Int J Cancer 57:353-361) gene amplification accounts for only 10% of these cases (Fantl, et al. (1993) Cancer Surv 18:77-94 (1993). Other mechanisms, such as up-regulation of gene transcription, must play a substantial role in the overexpression of cyclin D1. To determine whether Pin1 regulates transcription of cyclin D1, various cyclin D1 promoter-luciferase reporter constructs constructs (full-length “−1745” and activated ras-responsive “−964” of FIG. 5, see, e.g., Motokura and Arnold (1993) Genes Chromosomes Cancer 7:89-95, and Albanese et al., (1995) JBC 270:23589-23597) were transfected into HeLa and MCF-7 cells in order to measure the response to manipulating Pin1 function. The level of Pin1 in cells can be readily manipulated by expressing a sense or antisense Pin1 construct, respectively (see, e.g., Lu et al., (1996) Nature 380:544-547). FIG. 5 shows that both reporters were strongly transcribed in response to the expression of Pin1. Compared with the antisense construct, the Pin1 sense construct increased the activity of the cyclin D1 promoter by about 15 fold. These results indicate that Pin1 activates the cyclin D1 promoter and that the −964CD1 promoter fragment retains the complete responsiveness to Pin1. Similar promoter activation transfection experiments were conducted in inducible Pin1-expressing cells using the promoters for two genes associated with beta-catenin tumorigenic pathways (TCF-1 and c-myc) to drive luciferase expression. As with cyclin D1, Pin1 expression was able to induce these promoters as well.

FIG. 5 depicts how the −964CD1 promoter fragment (of the cyclin D1 gene) contains binding sites for various transcriptional factors including a CREB site, four TCF sites, three Ets sites and one AP-1 site. To determine which element in the promoter is necessary for the Pin1 responsiveness, two deletion constructs containing either 22 bp (“−22”) or 163 bp (“−163”) of the cyclin D1 promoter were created and subjected to similar transactivation assays. FIG. 5 shows that Pin1 did not have any significant transactivating effect either on the −22 or the −163 reporter. These results indicate that Pin1 does not affect the cyclin D1 promoter activity through the basic transcriptional machinery and suggest that the major sequences responsible for the Pin1 responsiveness may be the AP-1 site and/or Ets sites. To examine the importance of the AP-1 site, a mutant promoter, “−964 AP-1mt” which contains two base pair substitutions at the consensus AP-1 site was used (see, e.g., Albanese et al., supra). FIG. 5 shows that elimination of the AP-1 site completely abolished the ability of Pin1 to activate the cyclin D1 promoter. Interestingly, the same mutation has been shown also to completely abolish the Ras- or c-Jun-dependent activation of cyclin D1 expression. These results indicate that the AP-1 site is essential for activation of the cyclin D1 promoter by Pin1, as is by Ras- or c-Jun.

The AP-1 complex is composed of c-Jun and c-Fos proteins, with c-Jun being the most potent transactivator in the complex (see, e.g., Chiu et al (1989) Cell 59:979-986, Angel et al (1989) New Biol. 1 :35-43, Abate, et al (1991) Mol Cell Biol 11:3624-3632. Various oncoproteins, including activated Ras, participate in a signaling cascade leading to phosphorylation of c-Jun on two S-P motifs (S63/73-P) to increase its transcriptional activity towards its target genes, including cyclin D1. In fact, Ras-mediated tumorigenesis depends on signaling pathways that act preferentially through cyclin D1 (Robles, et al. (1998) Genes Dev 12:2469-2474). Since Pin1 binds and regulates the function of phosphoproteins, it is possible that Pin1 activates the cyclin D1 promoter via modulating the activity of phosphorylated c-Jun. This possibility was tested by examining whether Pin1 binds to phosphorylated c-Jun. To manipulate phosphorylation of c-Jun on S63/73-P, we co-transfected c-Jun with the oncogenic Harvey-Ras (Ha-Ras or RasL61), the dominant-negative Ras (DN-Ras or RasN17) or the control vector, and then examined the ability of c-Jun to bind Pin1 by subjecting cell lysates to GST-Pin1 pulldown experiments (see, e.g., Yaffe, et al. (1997) Science 278:1957-1960, Shen, et al (1998) Genes Dev. 12:706-720, Lu, et al. (1999) Science 283, 1325-1328). Although there was no binding at all between GST and c-Jun, weak binding between GST-Pin1 and c-Jun was detected when only c-Jun was transfected. Furthermore, the binding was significantly increased by co-transfection with Ha-Ras, but not with DN-Ras. Since Ha-Ras is known to induce phosphorylation of c-Jun on S63/73-P, the binding may be mediated by phosphorylation on these residues. To test this possibility, we used a c-Jun mutant (c-JunS63/73A; contains double Ala substitutions at S63 and S73, see, e.g., Smeal, et al (1991) Nature 354:494-496). Although the mutant was expressed at much higher levels and did not display a significant mobility shift, as compared with wild type protein, much less of the mutant protein was precipitated by Pin1. These results indicate that although the mutant c-JunS63/73A may contain some other minor Pin1 -binding site(s), phosphorylation of c-Jun on S63/73-P is important for the Pin1 binding. Thus, Pin1 binds to c-Jun mainly via phosphorylated S63/73-P motifs.

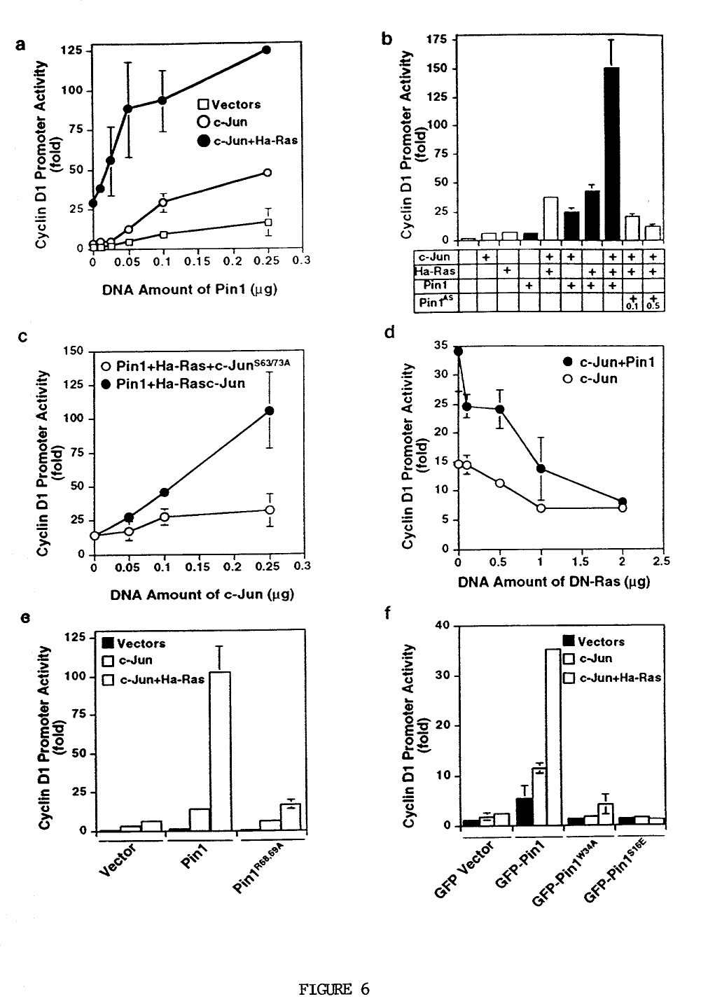

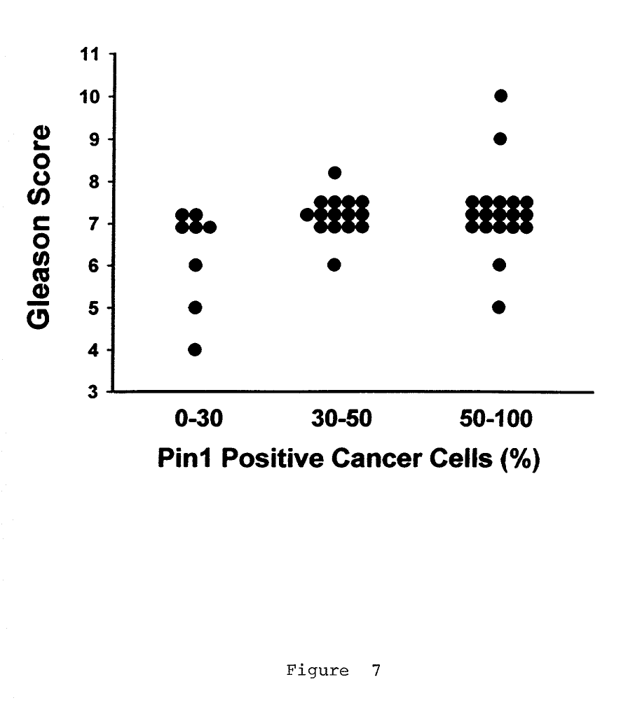

The ability of Pin1 to modulate the activity of c-Jun in activating the cyclin D1 promoter in presence or absence of activated Ras was next assessed. When Pin1 cDNA was co-transfected into HeLa cells with c-Jun, c-Jun and Ha-Ras or control vectors, Pin1 levels were slightly increased by co-transfection with c-Jun and further increased by co-transfection with c-Jun and Ha-Ras. These results indicate that Ha-Ras and c-Jun can increase the protein level of exogenously expressed Pin1. More importantly, although Pin1 did not affect levels of phosphorylated c-Jun in the presence or absence of Ha-Ras, Pin1 potently cooperated with c-Jun in activating the cyclin D1 promoter in a concentration-dependent manner (FIG. 6, panels “a” and “b”). The activity of the cyclin D1 promoter in cells co-transfected with Pin1 and c-Jun was 3-5 fold higher than that in cells transfected with either Pin1 or c-Jun alone. An even more dramatic potentiation of cyclin D1 reporter gene activity (by 5-10 fold) occurred if c-Jun was activated by Ha-Ras in the presence of Pin1. These results indicate that Pin1 and c-Jun cooperatively activate the cyclin D1 promoter and that this cooperation is further potentiated by oncogenic Ras.

The ability of Pin1 to activate the cyclin D1 promoter by modulating the activity of phosphorylated c-Jun was next assessed. To accomplish this, it was postulated that a mutation of the c-Jun phosphorylation sites would abolish the effect of Pin1 on the cyclin D1 promoter. The c-Jun^{S63/73A} mutant was used to examine this possibility. As shown in FIG. 6, panel “c”, Pin1 almost completely failed to cooperate with c-Jun^{S63/73}A to induce the cyclin D1 promoter. These results indicate that phosphorylation of c-Jun on S^{63/73} is essential for Pin1 to induce the cyclin D1 promoter. To further confirm this conclusion and to examine the importance of the Ras-dependent signaling in this regulation, we used DN-Ras to inhibit endogenous Ras function. DN-Ras not only inhibited the ability of c-Jun to activate the cyclin D1 promoter, but also potently inhibited the ability of Pin1 to enhance the activity of c-Jun in a concentration-dependent manner (FIG. 6, panel “d”). These results indicate a critical role of the Ras-dependent signaling for Pin1 to modulate c-Jun activity. These results together indicate that phosphorylation of c-Jun on S^{63/73} induced by the Ras-dependent signaling pathway is essential for Pin1 to modulate the transcriptional activity of the cyclin D1 promoter.

To examine whether the activities of the WW domain and a PPIase domain are required for Pin1 to modulate the activity of c-Jun, similar experiments were carried out with Pin1 mutants, Pin1^{R68,69A}, Pin1^{W34A} and Pin1 ^{S16E}, which contain mutations at the key residues either in the PPIase domain (R68, R69) or the WW domain (W34 or S16) and fail to isomerize pS/T-P bonds or to bind phosphoproteins. As shown in FIG. 6, panels “e” and “f”, these Pin1 mutants neither increased the transcriptional activity of c-Jun towards the cyclin D1 promoter, nor potentiated the ability of Ha-Ras to activate c-Jun. These results indicate that both phosphoprotein-binding and phosphorylation-specific isomerase activities are required for Pin1 to modulate the activity of c-Jun.

To examine whether endogenous Pin1 is important for activation of the cyclin D1 promoter by c-Jun and H-Ras, we again transfected the expression vector which contains antisense Pin1 (Pin1^{AS}) which significantly reduces cellular Pin1 levels. When c-Jun and H-Ras were cotransfected with different concentrations of the Pin1^{AS} construct, the cyclin D1 promoter activity was significantly decreased in a concentration-dependent manner (FIG. 6, panel “b”). Since depletion of Pin1 did not significantly affect levels of phosphorylated c-Jun, these results indicate that inhibiting endogenous Pin1 decreases the ability of phosphorylated c-Jun to activate the cyclin D1 promoter.

### Example 6

### Primary Screen for Pin1 Expression in Human Tissues

Materials and Methods

Human Tissue Samples

Formalin-fixed, paraffin-embedded sections of normal human organs were obtained from Novagen (Madison, Wis.). Organs examined included: prostate, brain, pituitary gland, kidney, muscle, esophagus, stomach, small intestines, colon, liver, spleen, pancreas, thyroid, heart, lung, bladder, adipose, lymph node, uterus, ovary, adrenal, testis, tonsil and thymus.

Formalin-fixed, paraffin-embedded sections of 19 different human tumor tissues were obtained from both Novagen and Imgenix (San Diego, Calif.). Cancers examined included: prostate, stomach, breast, pancrease, lung, liver, renal, ovary, thyroid, bladder, uterine cervix, colon, esophagus, lymphoma, endometrium, head/neck, gallbladder, melanoma, parotid.

Antibody

A commercial polyclonal antibody (Ab-1) (Oncogene Research Products, MA) was employed in this study, which was generated by immunizing rabbits with recombinant human Pin1. The specificity of the antibody was tested and confirmed by Western blotting and affinity purification.

Immunohistochemistry

Immunohistochemistry was performed on formalin-fixed tissues embedded in paraffin and sectioned at 4 to 6 μm for both normal and tumor tissues. The sections were deparaffinized in xylene, rehydrated in graded ethanols (100, 95 and 75%), followed by immersed in 3% H2O2/methanol for 15 minutes. For antigen retrieval, sections were microwaved in citrate buffer (pH 6.0) (BioGenex) for 15 minutes. Sections were then blocked in 10% normal goat serum in TBS, followed by incubation with primary antibody 1:800 overnight at 4° C. Incubation with biotinylated goat anti-rabbit antibody (Vector Laboratories, Burlingame, Calif.) for 30 minutes at room temperature was followed by the standard avidin-biotin-complex (ABC) process (Vectastain Elite ABC kit, Vector). Diaminobenzidine (DAB) was used as a chromogen, followed by counterstaining with hematoxylin. For negative controls, the primary antibody was omitted and prior immunostaining with preabsorbed antibody did not reveal any specific reactivity.

Results

Normal Tissues

Normal human tissue samples from 25 organs were studies. In normal tissues, the Pin1 level was low except for normal kidney, brain, pancreatic islet cells and testis tissues where higher levels of Pin1 were detected.

Tumor Tissues

260 tumor samples from 19 different types of common human cancers were studied. All 19 different types of cancers have shown Pin1 over-expression. The incidence of Pin1 protein over-expression varies in different types of cancers (Table 1).

TABLE 1
Pin1 Expression in Human Tumors

| Tumor Type | Total number | % Positive |
|---|---|---|
| Prostate | 49 | 92% |
| Stomach | 18 | 28% |
| Breast | 17 | 100% |
| Pancreatic | 16 | 33% |
| Lung | 14 | 50% |
| Liver | 13 | 31% |
| Renal | 13 | 23% |
| Ovary | 12 | 58% |
| Thyroid | 12 | 58% |
| Bladder | 11 | 81% |
| Uterine Cervix | 11 | 73% |
| Colon | 11 | 55% |
| Esophagus | 11 | 55% |
| Malignant lymphoma | 10 | 90% |
| Endometrium | 10 | 90% |
| Head/Neck | 10 | 60% |
| Gallbladder | 10 | 45% |
| Malignant melanoma | 9 | 100% |
| Parotid | 3 | 33% |

### Example 7

### Use of Pin1 as a Prognostic Marker in Human Prostate Cancer

Materials and Methods

A total of 42 patients with prostatic adenocarcinoma underwent radical prostatectomy between 1988 and 1996. The clinical stage of the prostate tumor was assessed retrospectively by a review of the medical records. The grade of each neoplasm was determined using the Gleason scoring system.

Antibody

A commercially available human polyclonal Pin1 antibody (Oncogene Research Products, MA) was used in this study. The antibody was affinity-purified using CNBr-activated Sepharose 4B column (Amersham Pharmacia Biotech). The purified antibody was tested on a Western blot which contained recombinant human Pin1 protein.

Immunohistochemical Staining.

Human prostate cancer sections were stained for Pin1 using an avidin-biotin-peroxidase complex (ABC) method (Vector, Burlingame, Calif.). Formalin-fixed, paraffin-embedded 5 μm tissue sections were deparaffinized in xylenes, rehydrated in graded alcohols, and blocked for endogenous peroxidase activity by 3% hydrogen peroxide (Sigma) in methanol for 15 min. For antigen retrieval, sections were microwaved in citrate buffer, pH 6.0 (BioGenex) for 15 min. The sections were then treated with 10% normal serum same specie as secondary antibody for 40 min to prevent nonspecific binding before incubating with an anti Pin1 antibody overnight at 4° C. at 1:800 (v/v) dilution. The sections were washed 4 times (5 min each) with TBS followed by incubation with a biotinylated goat anti-rabbit IgG antibody for 40 min. After incubation with a preformed avidin-biotin complex for 40 min, specifically bound antibodies were visualized by using peroxidase substrate, 3, 3′-diaminobenzidine tetrahydrochloride (DAB). Sections were counterstained with Gill's hematoxylin. Negative controls included sections without primary antibody or with normal serum instead of Pin1 antibody.

Discussion

Using affinity purified polyclonal Pin1 antibody, an immunohistochemical study on paraffin sections of 42 human prostate carcinoma cases was conducted. Positive immunostaining was observed in the cytoplasm as well as the nucleus of epithelial cells in neoplastic prostates but not in or very little in normal prostates. The stromal cells surrounding tumors showed no or very little Pin1 expression. Among the specimens investigated, well differentiated carcinomas with Gleason scores 4-5 generally showed no Pin1 staining or very low levels of staining. In some cases, high-grade prostatic interstitial neoplasia (PIN) showed Pin1 immunostaining, but usually to a lesser extent than the malignant lesions. Moderately differentiated prostate carcinomas with Gleason 6-7 showed partially positive immunostaining, in which not all cancer cells expressed Pin1, nor all cancerous lesions in a same specimen. Poorly differentiated prostate carcinomas with Gleason scores 8-10 displayed the most extensive and intense Pin1 immunoreaction.

Pin1 staining levels for all prostatic carcinoma specimens are summarized in FIG. 7. The results showed a general correlation between Pin1 expression and the Gleason scores, with high grade tumors (Gleason scores of 8-10) showing a higher percentage of positive staining than low grade (Gleason scores of 4-5) tumors. Interestingly, moderately differentiated prostate carcinomas with Gleason scores of 6-7 could be divided into three groups according to the levels of Pin1 expression. Group I: less than 30% of cancer cells in a whole section stained for Pin1; group II: 30-50% of cancer cells stained for Pin1; group III: more than 50% stained for Pin1. 8 out of 42 cases (19%) were classified as group I; 15 (36%) cases were classified as group II; and 19 (45%) were classified as group III.

Gleason grading system is the most common clinical practice for prostate cancer, with high Gleason scores showing high rate of recurrence and metastasis, and low Gleason scores showing low rate of mortality. Patients in intermediate grade (Gleason score 6-7) have various outcomes. Most people diagnosed as having prostate cancer belong to this group and present the biggest challenge to the diagnosing clinician. Patients in group I appear to represent an indolent disease course and have a low risk of developing metastatic disease; patients in group III are likely to go on to develop metastatic disease. Therefore, Pin1 staining of prostate cancer is a useful tool to measure the degree of biological aggressiveness of prostate cancer.

Clinical follow-up for three years or more on patients is summarized in Table 2. Prostate specific antigen (PSA) is commonly used for early diagnosis of prostate cancer and monitoring the effectiveness of treatment. After surgery, PSA level is undetectable. In the follow-up, if PSA level becomes detectable, it is called PSA failure that indicates either primary tumor recurrence or development of metastasis. Based on Pin1 expression and PSA follow-up, it was found that there is a tendency that at the time of surgery, patients whose tumor showed high levels of Pin1 expression were likely to experience PSA failure. Table 3 shows the correlation between Pin1 expression and PSA failure. Patients with low levels of Pin1 expression (0-30% of tumor cells positive) showed low rate of PSA failure (12.5%), followed by medium levels of Pin1 expression (30-50%) showing higher rate of failure (64%). Patients with high levels of Pin1 expression (50-100%) exhibited the highest rate of PSA failure (78%).

TABLE 2
Pin1 Expression and Clinical Outcome in Prostate Cancer

| | Pin1 | % Cells | Glea- | | PSA | PSA |
|---|---|---|---|---|---|---|
| | Inten- | Expressing | son | Surgery | Fail- | failure- |
| | sity | Pins | Sum | date | ure | Date |
| 1 | + | 50% | 7 | Sep. 6, 1991 | 1 | Aug. 24, 1992 |
| 2 | + | 50% | 4 | Jan. 24, 1992 | 0 | Mar. 30, 1995 |
| 3 | + | 80% | 7 | Aug. 17, 1992 | 0 | Sep. 16, 1992 |
| 4 | + | 90% | 10 | Oct. 20, 1992 | 1 | Jul. 1, 1993 |
| 5 | + | 40% | 7 | Nov. 11, 1992 | n/a | |
| 6 | ++ | 70% | 7 | Dec. 7, 1992 | 1 | Jul. 1, 1993 |
| 7 | + | 30% | 7 | Mar. 1, 1993 | 0 | Nov. 7, 1996 |
| 8 | + | 50% | 7 | May 14, 1993 | 0 | Aug. 8, 1996 |
| 9 | + | 60% | 7 | Sep. 28, 1993 | 1 | Dec. 12, 1995 |
| 10 | + | 70% | 8 | Nov. 7, 1994 | 1 | May 16, 1995 |
| 11 | + | 40% | 8 | Dec. 5, 1994 | 1 | Sep. 3, 1996 |
| 12 | ++ | 70% | 7 | Jan. 4, 1995 | 1 | Sep. 13, 1996 |
| 13 | + | 80% | 7 | Feb. 15, 1995 | 1 | Jul. 31, 1995 |
| 14 | + | 80% | 5 | Feb. 24, 1995 | 0 | Nov. 22, 1996 |
| 15 | + | 50% | 7 | Apr. 13, 1995 | 1 | Jun. 16, 1995 |
| 16 | + | 80% | 7 | Mar. 8, 1995 | 0 | May 21, 1997 |
| 17 | + | 30% | 7 | May 12, 1995 | 0 | Dec. 13, 1995 |
| 18 | + | 80% | 7 | May 9, 1995 | 0 | Oct. 24, 1996 |
| 19 | + | 40% | 7 | Sep. 22, 1995 | 0 | Apr. 11, 1997 |
| 20 | + | 80% | 8 | Jan. 29, 1996 | n/a | |
| 21 | ++ | 70% | 7 | Sep. 13, 1989 | 1 | Aug. 01, 1991 |
| 22 | + | 40% | 7 | Jan. 23, 1990 | 1 | Jul. 10, 1992 |
| 23 | ++ | 30% | 7 | Dec. 20, 1989 | 1 | Jul. 26, 1991 |
| 24 | + | 40% | 6 | Feb. 07, 1990 | 0 | 0 |
| 25 | + | 40% | 7 | May 6, 1988 | 0 | Feb. 11, 1993 |
| 26 | ++ | 70% | 9 | Nov. 17, 1988 | 1 | Sep. 10, 1990 |
| 27 | + | 40% | 7 | Feb. 13, 1990 | 1 | May 27, 1993 |
| 28 | ++ | 60% | 8 | Apr. 24, 1991 | 1 | Oct. 7, 1999 |
| 29 | + | 60% | 7 | Sept. 18, 1991 | 1 | May 12, 1995 |
| 30 | + | 50% | 7 | Oct. 21, 1991 | 0 | Oct. 25, 1999 |
| 31 | + | 50% | 6 | Oct. 25, 1991 | 1 | Jul. 16, 1992 |
| 32 | + | 30% | 5 | May 18, 1999 | 0 | Mar. 21, 1995 |
| 33 | + | 50% | 7 | Feb. 22, 1989 | 1 | Mar. 13, 1989 |
| 34 | + | 60% | 7 | Feb. 22, 1989 | 1 | Mar. 13, 1989 |
| 35 | + | 10% | 7 | May 24, 1989 | 0 | Jun. 14, 1999 |
| 36 | + | 70% | 7 | Jan. 2, 1990 | 0 | Jan. 17, 1992 |
| 37 | + | 50% | 7 | Feb. 16, 1990 | 1 | Nov. 15, 1990 |
| 38 | + | 70% | 7 | Nov. 8, 1990 | 1 | Sep. 11, 1991 |
| 39 | + | 60% | 7 | Dec. 14, 1990 | 1 | Sep. 27, 1995 |
| 40 | + | 50% | 7 | Feb. 27, 1991 | 1 | Jun. 11, 1992 |
| 41 | + | 10% | 6 | May 9, 1991 | 0 | Jul. 11, 1997 |
| 42 | + | 10% | 7 | Jul. 8, 1994 | 0 | Jul. 1, 1995 |

TABLE 3
Pin1 Expression and PSA Failure in Prostate Cancer

| % Pin1 positive | PSA Failure | | |
|---|---|---|---|
| cells | + | − | % |
| 0-30 | 1 | 7 | 12.5 |
| 30-50 | 9 | 5 | 64 |
| 50-100 | 14 | 4 | 78 |

As the above data show, patients with the most extensive Pin1 staining are at greater risk to develop recurrent disease than those with low Pin1 staining, and Pin1 can be used as a biomarker that functions as an indicator of metastatic progression and disease outcome in human prostate cancer patients.

### Example 8

### The use of Tissue Microarrys to Analyze Pin1 Expression in Human Tissue

Materials

Tissue Microarray:

A large human tissue microarray which included 2041 patients' tumor samples from 60 different tumor types and 229 normal samples from the matching normal organs was used in this study. All tissue samples were formalin-fixed and paraffin-embedded. H & E stained sections were made from each black to define representative tumor regions. Each tissue dot in the microarray was made in a diameter of 0.6 mm.

Methods:

1. Deparaffinzation. 4˜5 micron paraffin embedded sections are deparaffinized and hydrated:

| | |
|---|---|
| Zylenes | 10 min × 2 |
| 100% Ethanol | 5 min × 2 |
| 95% Ethanol | 5 min |
| 70% Ethanol | 5 min, |

Rinse sections in 3 changes of tap water

2. Endogenous peroxidases were blocked in 3% hydrogen peroxide in a methanol solution for 15 min at room temperature and rinsed with 3 changes of tap water.

3. Antigen retrieval. Slides were rinsed once in TBS buffer, transferred to a staining dish (VWR Scientific Products, West Chester, Pa., Cat. # 25608-906) which contained 200 ml of Antigen Retrieval Citra solution (pH6.0) (BioGenex, San Ramon, Calif., Cat. # HK086-9K). The staining dish was covered and placed in a microwave oven (Panasonic, Inverter, the Genius 1300W) at full power level to bring the solution to a boil (about 2 min). Once boiling, the power level of microwave was immediately reduced to the lowest point (10% of power) and the slides were heated for 15 min. The staining dish was removed from the microwave, and the slides allowwd to cool at room temperature for about 20 min. Slides were rinsed with 3 changes of TBS buffer.

4. As much of TBS as possible was carefully removed without allowing the sections to dry. 600 μl of normal goat serum (Vector Laboratories, Burlingame, Calif., Vectastain Elite ABC Kit, Rabbit IgG, Cat. # PK-6101)(5% in TBS) was added to each slide and incubated in a humidity container for 40 min at room temperature.

5. As much of normal goat serum as possible was carefully removed without allowing the sections to dry. 800 μl of Pin1 polyclonal antibody (Oncogene Research Products, Cambridge, Mass., Cat. # PC270, 4 mg/ml) was diluted 1:10,000 in TBS and dispensed onto each slide and incubated in a humidity container overnight at 4° C.

6. Slides were washed in 5 changes of TBS buffer for 5 min intervals.

7. As much of TBS as possible was carefully removed without allowing sections to dry. 600 μl of biotinylated anti rabbit IgG (Vector Laboratories, Vectastain Elite ABC Kit) diluted at 1:300 in 5% normal goat serum in TBS was added to each slide and incubated in a humidity container for 40 min at room temperature.

8. Slides were washed in 5 changes of TBS buffer for 5 min intervals.

9. As much of TBS as possible was carefully removed without allowing sections to dry. 600 μl of preformed ABC reagent (Vector Laboratories, Vectastain Elite ABC Kit Vector Laboratories, Vectastain Elite ABC Kit, prepared according to the instruction of the kit) was dispensed onto each slide and incubated in a humidity container for 40 min at room temperature.

10. Slides were washed in 5 changes of TBS buffer for 5 min intervals.

11. As much of TBS as possible was removed without allowing sections to dry. 600 μl of DAB solution (Vector Laboratories, Peroxidate Substrate Kit, Cat. # SK-4100) was dispensed onto each slide and incubated for 4-6 min at room temperature.

12. Substrate development was stopped by rinsing slides in running tap water.

13. Slides were counterstained in Mayer's Hematoxylin solution (VWR Scientific Products, Cat. # VW3414-1) for 40 seconds at room temperature, and rinsed in running tap water.

14. Samples were dehydrate in 70% Ethanol, 5 min, 95% Ethanol, 5 min, 100% Ethanol, 5 min×2, clear in Zylenes, 5 min, mounted in Permount (Fisher Scientific, Pittsburgh, Pa., cat. # SP15-100), and covered with coverglass.

Reagent Preparation

| 1. Tris Buffered Saline (TBS) pH 7.5 | | |
|---|---|---|
| 1) M Tris HCl, pH 7.5 (Gibco BRL, Cat. # 15567- | 10 | ml |
| 027) | | |
| Sodium chloride (Sigma, S-9888) | 10 | g |
| Nanopure water | final to 1000 | ml |
| 2. 3% Hydrogen peroxide | | |
| 30% Hydrogen peroxide (VWR, Cat. # VW3742-1) | 20 | ml |
| Methanol (VWR, Cat. # VW4325-4) | 180 | ml |
| 3. Antigen Retrieval Citra solution | | |
| Antigen Retrieval Citra solution (pH 6.0) (BioGenex, | 100 | ml |
| Cat. # HK086-9K) | | |
| Nanopure water | 900 | ml |

4. 3,3 diaminobenzidine (DAB) solution

Using the Peroxidase Substrate Kit (Vector Laboratories, Cat. # SK-4100)

| | |
|---|---|
| Tris buffer pH 7.4 | 2 drops |
| Hydrogen peroside | 2 drops |
| DAB solution | 4 drops |
| Nanopure water | 5 ml |

### Quantitative Evaluation of the Immunohistochemical Staining

1. Automated Cellular Imaging System (ACIS)

Each micro-histoarray section was scanned and images were captured using the automated cellular imaging system (ChromaVision Medical Systems, Inc., San Juan Capistrano, CA) which combines automated microscopy and computerized image processing to analyze of multiple tissues on a single slide. In this study, ACIS was used to analyze microarray tissue sections on glass slides stained using a diaminodenzidine chromagen (DAB) and hematoxylin counterstain. Positive staining (brown color) as viewed by light microscope indicates the presence of the protein, and color intensity correlates directly with protein quantity (expression). The ACIS was able to recognize 255 levels of immunohistochemical staining intensity (0-255) and converted these to fractional scores for the selected individual areas. However, the base limit on the threshold for the Generic DAB is pre-set at 50 by the manufacturer because the system is very sensitive. Therefore, any intensity below 50 was treated as 0 in this study. Entire immunostained tissue sections were scanned using the 4× objective and images were captured using the 10× objective.

2. Calculation of Pin Protein Expression in Human Cancers

In this study, we used the intensity scoring and the percent positive scoring (brown area was divided by total area) with the entire individual tissue dot selected. The immunohistochemical staining was quantitated without knowledge of the pathologist's score. All tissue samples were immunostained twice in University of Basel and in Pintex Pharmaceuticals, Inc. and the two data sets were evaluated in Pintex Pharmaceuticals, Inc. The final score was obtained by using the average of two data sets and calculated by the formulation:

score=intensity+(10×percent positive staining).

The % of total cases showing elevated levels (over-expression) of Pin1=numbers of tumor samples with score larger than the score of the highest normal case) total number of tumor samples

Results

Pin1 protein over-expression in human tissues microarray

| | | % of Tumor Cases with |
|---|---|---|
| Tumor type | Case number | Elevated Level of Pin1 |
| Brain tumor (3) | 111 | |
| Oligodendroglioma | 20 | 90 |
| Astrocytoma | 46 | 63 |
| Glioblastomamultiforme | 45 | 87 |
| Genecological tumor (13) | 372 | |
| Cervical carcinoma | 42 | 81 |
| Endometrium, endometroid | 46 | 0 |
| carcinoma | | |
| Endometrium, serous | 13 | 0 |
| carcinoma | | |
| Ovary, endometroid cancer | 45 | 24 |
| Ovary, Brenner tumor | 8 | 63 |
| Ovary mucinous cancer | 12 | 58 |
| Ovary, serous cancer | 47 | 43 |
| Uterus, carcinosarcoma | 6 | 100 |
| Breast, lobular cancer | 36 | 56 |
| Breast, ductal cancer | 47 | 47 |
| Breast, medullary cancer | 24 | 29 |
| Breast, mucinous cancer | 24 | 29 |
| Breast tubular cancer | 22 | 9 |
| Endocrine tumor (8) | 213 | |
| Thyroid adenocarcinoma | 42 | 29 |
| Thyroid follicular cancer | 49 | 41 |
| Thyroid medullary cancer | 8 | 100 |
| Thyroid papillary car | 36 | 22 |
| Parathyroid, adenocarcinoma | 28 | 21 |
| Adrenal gland adenoma | 15 | 0 |
| Adrenal gland cancer | 6 | 33 |
| Pheochromocytoma | 29 | 0 |
| Digestive tract tumor (11) | 411 | |
| Colon adenoma mild displasia | 47 | 21 |
| Colon adenoma moderate | 47 | 17 |
| displasia | | |
| Colon adenoma severe | 49 | 14 |
| displasia | | |
| Colon adenocarcinoma | 43 | 2 |
| Esophagus adenocarcinoma | 43 | 30 |
| Hepatocelluar carcinoma | 34 | 62 |
| Mouth cancer | 46 | 93 |
| Gall bladder adenocarcinoma | 28 | 14 |
| Pancreatic adenocarcinoma | 43 | 2 |
| Small intestine | 10 | 0 |
| adenocarcinoma | | |
| Stomach diffuse | 21 | 0 |
| adenocarcinoma | | |
| Genitourinary tract tumor (9) | 381 | |
| Prostate (hormone-refract) | 44 | 59 |
| Prostate (untreated) | 47 | 64 |
| Kidney chromophobic | 15 | 0 |
| carcinoma | | |
| Kidney clear cell carcinoma | 47 | 0 |
| Kidney oncocytoma | 8 | 0 |
| Kidney papillary carcinoma | 44 | 0 |
| Testis, non-seminomatous | 43 | 2 |
| cancer | | |
| Testis seminoma | 47 | 2 |
| Urinary bladder transitional | 86 | 2 |
| carcinoma | | |
| Respiratory tract tumor (4) | 184 | |
| Lung, adenocarcinoma | 44 | 27 |
| Lung, large cell cancer | 45 | 42 |
| Lung, small cell cancer | 47 | 57 |
| Lung, squmous cell | 48 | 44 |
| carcinoma | | |
| Hematological neoplasia (5) | 146 | |
| Hodgkin lymphoma | 23 | 0 |
| MALT lymphoma | 47 | 4 |
| NHL, diffuse large B | 22 | 18 |
| NHL, others | 30 | 23 |
| Thymoma | 24 | 8 |
| Skin tumor (5) | 178 | |
| Skin, malignant melanoma | 44 | 73 |
| Skin, basolioma | 44 | 39 |
| Skin, squamous cell | 39 | 13 |
| cancer | | |
| Skin, merkel zell cancer | 5 | 100 |
| Skin benign nevus | 46 | 52 |
| Soft tissue tumor (2) | 45 | |
| Lipoma | 25 | 20 |
| Liposarcoma | 20 | 75 |

### REFERENCES

Bailly et al. (1991) Nature 350:715-8

Blangy et al. (1995) Cell 83:1159-69

Bonnemann et al. (1996) Curr Opinion Pediatr 8:569-82. [a subsequent erratum appears in Curr Opinion Pediatr 1997 9:1961]

Bramblett et al. (1993) Neuron 10:1089-99

Clackson and Wells (1995) Science 267:383-6

Crenshawetal. (1998) EMBO J. 17:1315-27

Davis et al. (1983) Proc Natl Acad Sci USA 80:2926-2930

Dolinski and Heitman (1997) Peptidyl-prolyl isomerases (PPlases). In: Guidebook to Molecular Chaperones and Protein-Folding Catalysts, M.-J. eds. Getbing, Oxford University Press, pp.359-369

Dolinski et al. (1997) Proc. Natl. Acad. Sci. USA 94:13093-131098

Ermekova et al. (1998) Adv Exp Med Biol 446:161-80

Hanes et al. (1989) Yeast 5:55-72

Hani et al. (1995) Febs Lett 365:198-202

Heald and McKeon (1990) Cell 61:579-89

Huibregtse et al. (1995) Proc Natl Acad Sci USA 92:2563-7 [a subsequent erratum appears in Proc Natl Acad Sci USA 92:5249]

Hunter (1998) Cell 92:141-143

Izumi and Maller (1993) Mol Biol Cell 4:1337-50

King et al. (1994) Cell 79:563-571

Kops et al. (1998) J. Biol. Chem. 273:31971-6

Lu (1999) Prog. Cell Cycle Res. (in press)

Lu et al. (1996) Nature 380:544-7

Lu and Hunter (1995a) Cell 81:413-424

Lu and Hunter (1995b) Progress in Cell Cycle Research 1:187-205

Lu et al. (1999) Nature (in press)

Lu et al. (1998) Science 283:1325-1328

Macias et al. (1996) Nature 382:646-9

Maleszka et al. (1996) Proc Natl Acad Sci USA 93:447-51

Marchal et al. (1998) Mol Cell Biol 18:314-321

Mayer and Baltimore (1993) Trends Cell Biol 3:8-13

Mayer et al. (1995) Curr Biol 5:296-305

Meyn (1997) Oncology 11:349-56 (see also discussion 356, 361 and 365)

Muschel et al. (1997) Vitain Horm 53:1-25

Nefsky and Beach (1996) EMBO J. 15:1301-12

Nigg (1995) BioEssays 17:471-480

Nurse (1994) Cell 79:547-550

Pawson and Schlessinger (1993) Curr Biol 3:434-442

Pawson and Scott (1997) Science 278:2075-80

Piccart and Di Leo (1997) Semin Oncol 24:S10-27- S10-33

Rahfeld et al. (1994) FEBS Lett. 343:65-69

Ranganathan et al. (1997) Cell 89:875-886

Rotin (1998) Curr Top Microbiol Immunol 228:115-33

Sabo et al (1999) J Biol Chem 274:7952-7

Schlessinger et al (1995) Nature 373:536-9

Schreiber (1991) Science 251:283-7

Schutkowski et al. (1998) Biochemistry 3 7:5566-75

Shen et al. (1997) Proc. Natl. Acad. Sci. USA 94:13618-13623

Shen et al (1998) Genes Dev. 12:706-720

Staub et al. (1996) EMBO J. 15:2371-80

Stukenberg et al. (1997) Curr Biol 7:338-48

Sudol, M. (1996) Prog Biophys Mol Biol 65:113-32

Uchida et al (1999) FEBS Lett. 446:278-82

Winder (1997) J Muscle Res Cell Motil 18:617-29

Yaffe et al (1997) Science 278:1957-1960

Kuang et al. (1997) Science 278:1957-1960.

Yoshida and Ihara (1993) J Neurochern 61:1183-6

Young et al. (1994) Protein Sci 3:717-29

Zhou et al. (1995) Nature 373:536-9

### Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of detecting abnormal cell growth in a mammal, comprising assessing the level of Pin1 in a test sample from the mammal, wherein an elevation in the levels of Pin1 is indicative of abnormal cell growth.

2. The method of claim 1, wherein the level of Pin1 is a protein level.

3. The method of claim 1, wherein the level of Pin1 is a nucleic acid level.

4. The method of claim 1, wherein the test sample is a tissue sample.

5. The method of claim 4, wherein said tissue sample is selected from the group consisting of rectum, the brain, the mouth, central nervous system, breast tissue, the uterine cervix, the endometrium, the head/neck, the skin, parotid tissue, the prostate, the brain, the gall bladder, the esophagus, the colon, the lung, thyroid tissue, parathyroid tissue, the uterus, ovarian tissue, adrenal tissue, and testicular tissue.

6. The method of claim 1, wherein the test sample is a body fluid test sample selected from the group consisting of blood, ascites, serum, semen, prostate fluid, seminal fluid, urine, saliva, sputum, phlegm, pus, mucus, bone marrow, lymph, tears or brain body fluid test sample.

7. The method of claim 1 wherein, detecting the abnormal cell growth in a mammal, comprises the steps of:
detecting a level of Pin1 in a test sample; and
comparing the level of Pin1 in the test sample with a control level, and wherein a difference in the level of Pin1 in the test sample is indicative of abnormal cell growth in the mammal.

8. The method of claim 1, wherein the abnormal cell growth is cancer.

9. The method of claim 8, wherein the cancer is selected from the group consisting of oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, and liposarcoma.

10. A method of claim 2 wherein, the method of detecting the level of Pin1 protein in a test sample from a mammal, comprises the steps of:
contacting the test sample with an antibody having specificity for Pin1 under conditions suitable for binding of the antibody to Pin1 thereby resulting in the formation of a complex between the antibody and Pin1;
detecting the complex between the antibody and Pin1; and
comparing the amount of the complex in the test sample with an amount of a complex in a control sample, wherein an elevation in the amount of the complex between the antibody and Pin1 in the test sample compared to the complex in the control sample is indicative of abnormal cell growth.

11. The method of claim 10, wherein the antibody is a polyclonal antibody.

12. The method of claim 10, wherein the antibody is a monoclonal antibody.

13. The method of claim 10, wherein the antibody is detectably labeled.

14. The method of claim 13, wherein the detectable label is selected from the group consisting of a radioactive, enzymatic, biotinylated and fluorescent label.

15. The method of claim 10, wherein the complex is detected by incubating the complex with a second antibody specific for the complex, wherein the secondary antibody comprises a detectable label.

16. The method of claim 3, wherein the method further comprises the step of performing a polymerase chain reaction with oligonucleotide primers capable of amplifying the Pin1 nucleic acid prior to detection.

17. The method of claim 3, wherein the method further comprises the steps of:
contacting a test sample obtained from the mammal with a nucleic acid probe to a Pin1 nucleic acid;
maintaining the test sample and the nucleic acid probe under conditions suitable for a hybridization;
detecting the hybridization between the test sample and the nucleic acid probe; and
comparing the hybridization in the test sample from the mammal to a control test sample without the cellular proliferation, wherein an elevation in the hybridization signal in the test sample from the mammal compared to the control sample is indicative of abnormal cell growth.

18. A method of determining the stage of cancer in a test sample from a mammal, comprising assessing a level of Pin1 in the test sample, wherein the level of Pin1 correlates with the stage of the cancer.

19. The method of claim 18, wherein the level of Pin1 is a protein level.

20. The method of claim 18, wherein the level of Pin1 is a nucleic acid level.

21. The method of claim 18 wherein, detecting the abnormal cell growth in a mammal, comprises the steps of:
detecting a level of Pin1 in a test sample; and
comparing the level of Pin1 in the test sample with a control level, and wherein a difference in the level of Pin1 in the test sample is indicative of abnormal cell growth in the mammal.

22. A method of claim 18 wherein, the method of detecting the level of Pin1 protein in a test sample from a mammal, comprises the steps of:
contacting the test sample with an antibody having specificity for Pin1 under conditions suitable for binding of the antibody to Pin1 thereby resulting in the formation of a complex between the antibody and Pin1;
detecting the complex between the antibody and Pin1; and
comparing the amount of the complex in the test sample with an amount of a complex in a control sample, wherein an elevation in the amount of the complex between the antibody and Pin1 in the test sample compared to the complex in the control sample is indicative of abnormal cell growth.

23. The method of claim 22, wherein the antibody is a polyclonal antibody.

24. The method of claim 22, wherein the antibody is a monoclonal antibody.

25. The method of claim 22, wherein the antibody is detectably labeled.

26. The method of claim 18, wherein the method further comprises the step of performing a polymerase chain reaction with oligonucleotide primers capable of amplifying the Pin1 nucleic acid prior to detection.

27. The method of claim 18, wherein the method further comprises the steps of:
contacting a test sample obtained from the mammal with a nucleic acid probe to a Pin1 nucleic acid;
maintaining the test sample and the nucleic acid probe under conditions suitable for a hybridization;
detecting the hybridization between the test sample and the nucleic acid probe; and
comparing the hybridization in the test sample from the mammal to a control test sample without the cellular proliferation, wherein an elevation in the hybridization signal in the test sample from the mammal compared to the control sample is indicative of abnormal cell growth.

28. The method of claim 18, wherein the cancer is selected from the group consisting of oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, and liposarcoma.

29. A method of claim 18 wherein the stage of cancer is determined by assessing a level of a Pin1 nucleic acid in a test sample, comprising the steps of:
performing a polymerase chain reaction with oligonucleotide primers capable of amplifying the Pin1 nucleic acid;
detecting a level of amplified nucleic acid fragments of the Pin1 nucleic acid; and
comparing the level of amplified nucleic acid fragments in the test sample to a sample comprising varying stages of the abnormal cell growth, wherein the stage of abnormal cell growth in the mammal is determined.

30. A method of claim 18 wherein the stage of cancer is determined by assessing a level of a Pin1 nucleic acid in a test sample, comprising the steps of:
contacting a test sample obtained from the mammal with a nucleic acid probe to a Pin1 nucleic acid;
maintaining the test sample and the nucleic acid probe under conditions suitable for hybridization of the probe to Pin1 nucleic in the sample;
detecting the hybridization between the Pin1 nucleic acid of the test sample and the nucleic acid probe; and
comparing the hybridization in the test sample from the mammal to hybridization of the nucleic probe to Pin1 in a control, wherein the control sample comprises varying stages of the abnormal cell growth, thereby determining the stage of the abnormal cell growth in the mammal.

31. A method of evaluating the efficacy of a treatment of abnormal cell growth in a mammal, comprising comparing a level of Pin1 in at least two test samples, wherein the test samples comprise a first test sample obtained at a first time and a second test sample obtained at a later second time, wherein a decrease in the level of Pin1 between the two test samples indicates the efficacy of the treatment of the abnormal cell growth in the mammal.

32. The method of claim 31, wherein the level of Pin1 is a protein level.

33. The method of claim 31, wherein the level of Pin1 is a nucleic acid level.

34. The method of claim 31 wherein, detecting the abnormal cell growth in a mammal, comprises the steps of:
detecting a level of Pin1 in a test sample; and
comparing the level of Pin1 in the test sample with a control level, and wherein a difference in the level of Pin1 in the test sample is indicative of abnormal cell growth in the mammal.

35. A method of claim 31 wherein, the method of detecting the level of Pin1 protein in a test sample from a mammal, comprises the steps of:
contacting the test sample with an antibody having specificity for Pin1 under conditions suitable for binding of the antibody to Pin1 thereby resulting in the formation of a complex between the antibody and Pin1;
detecting the complex between the antibody and Pin1; and
comparing the amount of the complex in the test sample with an amount of a complex in a control sample, wherein an elevation in the amount of the complex between the antibody and Pin1 in the test sample compared to the complex in the control sample is indicative of abnormal cell growth.

36. The method of claim 31, wherein the antibody is a polyclonal antibody.

37. The method of claim 31, wherein the antibody is a monoclonal antibody.

38. The method of claim 31, wherein the antibody is detectably labeled.

39. The method of claim 31, wherein the method further comprises the step of performing a polymerase chain reaction with oligonucleotide primers capable of amplifying the Pin1 nucleic acid prior to detection.

40. The method of claim 31, wherein the method further comprises the steps of:
contacting a test sample obtained from the mammal with a nucleic acid probe to a Pin1 nucleic acid;
maintaining the test sample and the nucleic acid probe under conditions suitable for a hybridization;
detecting the hybridization between the test sample and the nucleic acid probe; and
comparing the hybridization in the test sample from the mammal to a control test sample without the cellular proliferation, wherein an elevation in the hybridization signal in the test sample from the mammal compared to the control sample is indicative of abnormal cell growth.

41. A kit for determining the level of Pin1 in a test sample from a mammal comprising one or more reagents for detecting Pin1.

42. The Kit of claim 41, wherein one of the reagents is an antibody.

43. The kit of claim 41, wherein one of the reagents is a DNA probe.

44. The kit of claim 41, wherein one of the reagents is a control.

45. A method for determining whether a subject having cancer is likely to respond to treatment comprising a Pin1 inhibitor compound, the method comprising:
assessing the level of Pin1 in a test sample from the subject; and
comparing the level of Pin1 in the test sample to the level of Pin1 in normal tissue, whereby an increased level of Pin1 in the test sample is indicative that the subject is likely to respond to treatment comprising a Pin1 inhibitor compound.

46. The method of claim 45 wherein the cancer is selected from the group consisting of oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, and liposarcoma.

47. A method of treating an individual suffering from cancer comprising, administering to said individaul a Pin1 inhibitor such that treatment occurs.

48. The method of claim 47 wherein said cancer is selected from the group consisting of oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kideny chromophobic carcinoma, kidney clear cell carcinoma, kidney oncocytoma, kideny papillary carcinoma, testis non-seminomatous cancer, testis seminoma, urinary bladder transitional carcinoma, lung adenocarcinoma, lung large cell cancer, lung small cell cancer, lung squmous cell carcinoma, Hodgkin lymphoma, MALT lymphoma, non-hodgkins lymphoma (NHL) diffuse large B, NHL, thymoma, skin malignant melanoma, skin basolioma, skin squamous cell cancer, skin merkel zell cancer, skin benign nevus, lipoma, and liposarcoma.
